SEQUENCE LISTING (52 sequences)

SEQ ID NO: 1 - DNA, 1470 bp, Bos taurus
atgggcgaccgcggcggcgcgggcggctcccggcgccggaggacggggtcgcggccttcgatccagggcggcagtgggcccgcggcagcggaagaggaggtgcgggatgtgggcgccggaggggacgcgccggtccgggacacagacaaggacggagacgtagacgtgggcagcggccactgggacctgaggtgtcaccgcctgcaggattccctgttcagttctgacagtggcttcagcaactaccgtggcatcctgaattggtgtgtggtgatgctgatcttaagcaacgcacggttatttctagagaacctcatcaagtatggcatcctggtggaccccatccaggtggtgtctctgttcctgaaggacccctacagctggccagctctgtgcctggtcattgtggccaatatctttgccgtggctgcgttccaggtggagaagcgcctggccgtgggagctctgacggagcaggcggggctgctgctgcacggggtcaacctggccaccattctctgcttcccagcggccgtggcctttctcctcgagtctatcactccagtgggctccgtgctggccctgatggtctacaccatcctcttcctcaagctgttctcctaccgggacgtcaacctctggtgccgagagcgcagggctggggccaaggccaaggctgctttggcaggtaaggcggccaacgggggagctgcccagcgcaccgtgagctaccccgacaacctgacctaccgcgatctctactacttcctcttcgcccccaccctgtgctacgagctcaacttcccccgctccccccgcatccgaaagcgcttcctgctgcggcgactcctggagatgctgttcctcacccagctccaggtggggctgatccagcagtggatggtcccggccatccagaactccatgaagcccttcaaggacatggactactcccgcatcgtggagcgcctcctgaagctggcggtccccaaccacctcatctggctcatcttcttctactggctcttccactcctgcctgaacgccgtggctgagctcatgcagtttggagaccgcgagttctaccgggactggtggaactccgagtccatcacctacttctggcagaactggaacatccctgttcacaagtggtgcatcagacacttctacaagcccatgctccggcggggcagcagcaagtgggcagccaggacggcagtgtttctggcctccgccttcttccacgagtacctggtgagcatccccctgcgcatgttccgcctctgggccttcaccggcatgatggcgcagatcccgctggcctggatagtgggccgcttcttccgcggcaactacggcaacgcggccgtgtggctgtcactcatcatcgggcagccggtggccgtcctgatgtacgtccacgactactacgtgctcaaccgtgaggcgccggcagccggcacctga

SEQ ID NO: 2 - DNA, 1470 bp, Bos taurus
atgggcgaccgcggcggcgcgggcggctcccggcgccggaggacggggtcgcggccttcgatccagggcggcagtgggcccgcggcagcggaagaggaggtgcgggatgtgggcgccggaggggacgcgccggtccgggacacagacaaggacggagacgtagacgtgggcagcggccactgggacctgaggtgtcaccgcctgcaggattccctgttcagttctgacagtggcttcagcaactaccgtggcatcctgaattggtgtgtggtgatgctgatcttaagcaacgcacggttatttctagagaacctcatcaagtatggcatcctggtggaccccatccaggtggtgtctctgttcctgaaggacccctacagctggccagctctgtgcctggtcattgtggccaatatctttgccgtggctgcgttccaggtggagaagcgcctggccgtgggagctctgacggagcaggcggggctgctgctgcacggggtcaacctggccaccattctctgcttcccagcggccgtggcctttctcctcgagtctatcactccagtgggctccgtgctggccctgatggtctacaccatcctcttcctcaagctgttctcctaccgggacgtcaacctctggtgccgagagcgcagggctggggccaaggccaaggctgctttggcaggtaaggcggccaacgggggagctgcccagcgcaccgtgagctaccccgacaacctgacctaccgcgatctctactacttcctcttcgcccccaccctgtgctacgagctcaacttcccccgctccccccgcatccgaaagcgcttcctgctgcggcgactcctggagatgctgttcctcacccagctccaggtggggctgatccagcagtggatggtcccggccatccagaactccatgaagcccttcaaggacatggactactcccgcatcgtggagcgcctcctgaagctggcggtccccaaccacctcatctggctcatcttcttctactggctcttccactcctgcctgaacgccgtggctgagctcatgcagtttggagaccgcgagttctaccgggactggtggaactccgagtccatcacctacttctggcagaactggaacatccctgttcacaagtggtgcatcagacacttctacaagcccatgctccggcggggcagcagcaagtgggcagccaggacggcagtgtttctggcctccgccttcttccacgagtacctggtgagcatccccctgcgcatgttccgcctctgggccttcaccggcctgatggcgcagatcccgctggcctggatagtgggccgcttcttccgcggcaactacggcaacgcggccgtgtggctgtcactcatcatcgggcagccggtggccgtcctgatgtacgtccacgactactacgtgctcaaccgtgaggcgccggcagccggcacctga

SEQ ID NO: 3 - protein, 489 aa, Bos taurus
MGDRGGAGGSRRRRTGSRPSIQGGSGPAAAEEEVRDVGAGGDAPVRDTDKDGDVDVGSGHWDLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKAANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWCIRHFYKPMLRRGSSKWAARTAVFLASAFFHEYLVSIPLRMFRLWAFTGMMAQIPLAWIVGRFFRGNYGNAAVWLSLIIGQPVAVLMYVHDYYVLNREAPAAGT

SEQ ID NO: 4 - protein, 489 aa, Bos taurus
MGDRGGAGGSRRRRTGSRPSIQGGSGPAAAEEEVRDVGAGGDAPVRDTDKDGDVDVGSGHWDLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKAANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWCIRHFYKPMLRRGSSKWAARTAVFLASAFFHEYLVSIPLRMFRLWAFTGLMAQIPLAWIVGRFFRGNYGNAAVWLSLIIGQPVAVLMYVHDYYVLNREAPAAGT

SEQ ID NO: 5 - DNA, 30 bp, Artificial Sequence
  Oligonucleotide Primer Sequence
acgttggatgtcagtacctggtgagcatcc

SEQ ID NO: 6 - DNA, 29 bp, Artificial Sequence
  Oligonucleotide Primer Sequence
acgttggatgtccagggctgctcacctgc

SEQ ID NO: 7 - DNA, 17 bp, Artificial Sequence
  Oligonucleotide Primer Sequence
gctcacctgcgccatca

SEQ ID NO: 8 - DNA, 19 bp, Artificial Sequence
  Oligonucleotide Primer Sequence (exon 1)
ggcggggtgcgaactaagg

SEQ ID NO: 9 - DNA, 18 bp, Artificial Sequence
  Oligonucleotide Primer Sequence (Exon 1)
ggccgctgcccacgtcta

SEQ ID NO: 10 - DNA, 20 bp, Artificial Sequence
  Oligonucleotide Primer Sequence (Exon 1)
gcggaagaggaggtgcggga

SEQ ID NO: 11 - DNA, 18 bp, Artificial Sequence
  Oligonucleotide Primer Sequence (Exon 1)
agcccgagctgccgccga

SEQ ID NO: 12 - DNA, 19 bp, Artificial Sequence
  Oligonucleotide Primer Sequence (Exon 2)
gccttgctgccagatgctc

SEQ ID NO: 13 - DNA, 20 bp, Artificial Sequence
  Oligonucleotide Primer Sequence (Exon 2)
agaccccagctcagaggctc

SEQ ID NO: 14 - DNA, 20 bp, Artificial Sequence
  Oligonucleotide Primer Sequence (Exon 3)
ggtggggagtctcagagctt

SEQ ID NO: 15 - DNA, 20 bp, Artificial Sequence
  Oligonucleotide Primer Sequence (Exon 3)
aggatgccatacctgcagac

SEQ ID NO: 16 - DNA, 25 bp, Artificial Sequence
  Oligonucleotide Primer Sequence (Exons 4-17)
ttaagcaacgcacggttatttctag

SEQ ID NO: 17 - DNA, 25 bp, Artificial Sequence
  Oligonucleotide Primer Sequence (Exons 4-17)
tgcacagcactttattgacacattc

SEQ ID NO: 18 - DNA, 25 bp, Artificial Sequence
  Oligonucleotide Primer Sequence
ttaagcaacgcacggttatttctag

SEQ ID NO: 19 - DNA, 25 bp, Artificial Sequence
  Oligonucleotide Primer Sequence
ggccgctgggaagcagagaatggtg

SEQ ID NO: 20 - DNA, 25 bp, Artificial Sequence
  Oligonucleotide Primer Sequence
cgtggctgcgttccaggtggagaag

SEQ ID NO: 21 - DNA, 25 bp, Artificial Sequence
  Oligonucleotide Primer Sequence
gtagcacagggtgggggcgaagagg

SEQ ID NO: 22 - DNA, 25 bp, Artificial Sequence
  Oligonucleotide Primer Sequence
gtgagctaccccgacaacctgacct

SEQ ID NO: 23 - DNA, 25 bp, Artificial Sequence
  Oligonucleotide Primer Sequence
ccagcttcaggaggcgctccacgat

SEQ ID NO: 24 - DNA, 20 bp, Artificial Sequence
  Oligonucleotide Primer Sequence
gctttttgcagtggatggtc

SEQ ID NO: 25 - DNA, 20 bp, Artificial Sequence
  Oligonucleotide Primer Sequence
gatgagccagatgaggtggt

SEQ ID NO: 26 - DNA, 20 bp, Artificial Sequence
  Oligonucleotide Primer Sequence
acatggactactcccgcatc

SEQ ID NO: 27 - DNA, 20 bp, Artificial Sequence
  Oligonucleotide Primer Sequence
acctgatgcaccacttgtga

SEQ ID NO: 28 - DNA, 20 bp, Artificial Sequence
  Oligonucleotide Primer Sequence
tgagctcatgcagtttggag

SEQ ID NO: 29 - DNA, 21 bp, Artificial Sequence
  Oligonucleotide Primer Sequence
gagcatgggcttgtagaagtg

SEQ ID NO: 30 - DNA, 20 bp, Artificial Sequence
  Oligonucleotide Primer Sequence
cctgttcacaagtggtgcat

SEQ ID NO: 31 - DNA, 20 bp, Artificial Sequence
  Oligonucleotide Primer Sequence
caccaggtactgaggggaga

SEQ ID NO: 32 - DNA, 20 bp, Artificial Sequence
  Oligonucleotide Primer Sequence
ccttcttccacgaggtcagt

SEQ ID NO: 33 - DNA, 20 bp, Artificial Sequence
  Oligonucleotide Primer Sequence
tcgtggacgtacatcaggac

SEQ ID NO: 34 - DNA, 25 bp, Artificial Sequence
  Oligonucleotide Primer Sequence
ggccgtgtggctgtcactcatcatc

SEQ ID NO: 35 - DNA, 25 bp, Artificial Sequence
  Oligonucleotide Primer Sequence
tgcacagcactttattgacacattc

SEQ ID NO: 36 - DNA, 18 bp, Artificial Sequence
  Oligonucleotide Primer Sequence
actaaggccatgggcgac

SEQ ID NO: 37 - DNA, 15 bp, Artificial Sequence
  Oligonucleotide Primer Sequence
ggggccagcctggag

SEQ ID NO: 38 - DNA, 32 bp, Artificial Sequence
  Oligonucleotide Primer Sequence
gggcgggatccaagcttatgggcgaccgcggc

SEQ ID NO: 39 - DNA, 39 bp, Artificial Sequence
  Oligonucleotide Primer Sequence
ccccgaattcgcggccgccagctgtcaggtgccggctgc

SEQ ID NO: 40 - DNA, 39 bp, Artificial Sequence
  Oligonucleotide Primer Sequence
ccccgaattcgcggccgcgtcgactcaggtgccggctgc

SEQ ID NO: 41 - DNA, 21 bp, Artificial Sequence
  Oligonucleotide Primer Sequence
cacttctacaagcccatgctc

SEQ ID NO: 42 - DNA, 16 bp, Artificial Sequence
  Oligonucleotide Primer Sequence
ttgccgtagttgccgc

SEQ ID NO: 43 - DNA, 1470 bp, Bos taurus
atgggcgaccgcggcggcgcgggcggctcccggcgccggaggacggggtcgcggccttcgatccagggcggcagtgggcccgcggcagcggaagaggaggtgcgggatgtgggcgccggaggggacgcgccggtccgggacacagacaaggacggagacgtagacgtgggcagcggccactgggacctgaggtgtcaccgcctgcaggattccctgttcagttctgacagtggcttcagcaactaccgtggcatcctgaattggtgtgtggtgatgctgatcttaagcaacgcacggttatttctagagaacctcatcaagtatggcatcctggtggaccccatccaggtggtgtctctgttcctgaaggacccctacagctggccagctctgtgcctggtcattgtggccaatatctttgccgtggctgcgttccaggtggagaagcgcctggccgtgggagctctgacggagcaggcggggctgctgctgcacggggtcaacctggccaccattctctgcttcccagcggccgtggcctttctcctcgagtctatcactccagtgggctccgtgctggccctgatggtctacaccatcctcttcctcaagctgttctcctaccgggacgtcaacctctggtgccgagagcgcagggctggggccaaggccaaggctgctttggcaggtaagaaggccaacgggggagctgcccagcgcaccgtgagctaccccgacaacctgacctaccgcgatctctactacttcctcttcgcccccaccctgtgctacgagctcaacttcccccgctccccccgcatccgaaagcgcttcctgctgcggcgactcctggagatgctgttcctcacccagctccaggtggggctgatccagcagtggatggtcccggccatccagaactccatgaagcccttcaaggacatggactactcccgcatcgtggagcgcctcctgaagctggcggtccccaaccacctcatctggctcatcttcttctactggctcttccactcctgcctgaacgccgtggctgagctcatgcagtttggagaccgcgagttctaccgggactggtggaactccgagtccatcacctacttctggcagaactggaacatccctgttcacaagtggtgcatcagacacttctacaagcccatgctccggcggggcagcagcaagtgggcagccaggacggcagtgtttctggcctccgccttcttccacgagtacctggtgagcatccccctgcgcatgttccgcctctgggccttcaccggcatgatggcgcagatcccgctggcctggatagtgggccgcttcttccgcggcaactacggcaacgcggccgtgtggctgtcactcatcatcgggcagccggtggccgtcctgatgtacgtccacgactactacgtgctcaaccgtgaggcgccggcagccggcacctga

SEQ ID NO: 44 - DNA, 1470 bp, Bos taurus
atgggcgaccgcggcggcgcgggcggctcccggcgccggaggacggggtcgcggccttcgatccagggcggcagtgggcccgcggcagcggaagaggaggtgcgggatgtgggcgccggaggggacgcgccggtccgggacacagacaaggacggagacgtagacgtgggcagcggccactgggacctgaggtgtcaccgcctgcaggattccctgttcagttctgacagtggcttcagcaactaccgtggcatcctgaattggtgtgtggtgatgctgatcttaagcaacgcacggttatttctagagaacctcatcaagtatggcatcctggtggaccccatccaggtggtgtctctgttcctgaaggacccctacagctggccagctctgtgcctggtcattgtggccaatatctttgccgtggctgcgttccaggtggagaagcgcctggccgtgggagctctgacggagcaggcggggctgctgctgcacggggtcaacctggccaccattctctgcttcccagcggccgtggcctttctcctcgagtctatcactccagtgggctccgtgctggccctgatggtctacaccatcctcttcctcaagctgttctcctaccgggacgtcaacctctggtgccgagagcgcagggctggggccaaggccaaggctgctttggcaggtaagaaggccaacgggggagctgcccagcgcaccgtgagctaccccgacaacctgacctaccgcgatctctactacttcctcttcgcccccaccctgtgctacgagctcaacttcccccgctccccccgcatccgaaagcgcttcctgctgcggcgactcctggagatgctgttcctcacccagctccaggtggggctgatccagcagtggatggtcccggccatccagaactccatgaagcccttcaaggacatggactactcccgcatcgtggagcgcctcctgaagctggcggtccccaaccacctcatctggctcatcttcttctactggctcttccactcctgcctgaacgccgtggctgagctcatgcagtttggagaccgcgagttctaccgggactggtggaactccgagtccatcacctacttctggcagaactggaacatccctgttcacaagtggtgcatcagacacttctacaagcccatgctccggcggggcagcagcaagtgggcagccaggacggcagtgtttctggcctccgccttcttccacgagtacctggtgagcatccccctgcgcatgttccgcctctgggccttcaccggcctgatggcgcagatcccgctggcctggatagtgggccgcttcttccgcggcaactacggcaacgcggccgtgtggctgtcactcatcatcgggcagccggtggccgtcctgatgtacgtccacgactactacgtgctcaaccgtgaggcgccggcagccggcacctga

SEQ ID NO: 45 - protein, 489 aa, Bos taurus
MGDRGGAGGSRRRRTGSRPSIQGGSGPAAAEEEVRDVGAGGDAPVRDTDKDGDVDVGSGHWDLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKKANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWCIRHFYKPMLRRGSSKWAARTAVFLASAFFHEYLVSIPLRMFRLWAFTGMMAQIPLAWIVGRFFRGNYGNAAVWLSLIIGQPVAVLMYVHDYYVLNREAPAAGT

SEQ ID NO: 46 - protein, 489 aa, Bos taurus
MGDRGGAGGSRRRRTGSRPSIQGGSGPAAAEEEVRDVGAGGDAPVRDTDKDGDVDVGSGHWDLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKKANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWCIRHFYKPMLRRGSSKWAARTAVFLASAFFHEYLVSIPLRMFRLWAFTGLMAQIPLAWIVGRFFRGNYGNAAVWLSLIIGQPVAVLMYVHDYYVLNREAPAAGT

SEQ ID NO: 47 - protein, 468 aa, Bos taurus
MGDRGGAGGSRRRRTGSRPSIQGGSGPAAAEEEVRDVGAGGDAPVRDTDKDGDVDVGSGHWDLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKAANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWCIRHFYKPMLRRGSSKWAARTAVFLASAFFHEIPLAWIVGRFFRGNYGNAAVWLSLIIGQPVAVLMYVHDYYVLNREAPAAGT

SEQ ID NO: 48 - protein, 468 aa, Bos taurus
MGDRGGAGGSRRRRTGSRPSIQGGSGPAAAEEEVRDVGAGGDAPVRDTDKDGDVDVGSGHWDLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKKANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWCIRHFYKPMLRRGSSKWAARTAVFLASAFFHEIPLAWIVGRFFRGNYGNAAVWLSLIIGQPVAVLMYVHDYYVLNREAPAAGT

SEQ ID NO: 49 - DNA, 20 bp, Artificial Sequence
  Oligonucleotide Primer
ctgttcctgaaggaccccta

SEQ ID NO: 50 - DNA, 18 bp, Artificial Sequence
  Oligonucleotide Primer
gcagccacggcaaagata

SEQ ID NO: 51 - DNA, 20 bp, Artificial Sequence
  Oligonucleotide Primer
ttgggttacaccgaagatgc

SEQ ID NO: 52 - DNA, 20 bp, Artificial Sequence
  Oligonucleotide Primer
agatggaagcgtgagtgtca

(19) United States
(12) Patent Application Publication
(10) Pub. No.: US 2011/0321182 A1
(43) Pub. Date: Dec. 29, 2011

(54) SELECTION OF ANIMALS FOR DESIRED MILK AND/OR TISSUE PROFILE

(76) Inventors: Klaus Lehnert, Auckland (NZ); Sarah Dianne Berry, Auckland (NZ); Russell Grant Snell, Auckland (NZ); Alastair Kenneth Hugh MacGibbon, Auckland (NZ); Richard Spelman, Auckland (NZ); Alexandra Ankersmit-Udy, Auckland (NZ)

(21) Appl. No.: 13/142,092

(22) PCT Filed: Dec. 24, 2009
(86) PCT No.: PCT/NZ2009/000296

§ 371 Date: Sep. 7, 2011

(30) Foreign Application Priority Data
Dec. 24, 2008 (NZ) 573950

(51) Int. Cl. A01K 67/027; C12Q 1/48; C12Q 1/68; C07H 21/04; C12N 9/10

(52) U.S. Cl. 800/13; 536/23.2; 435/193; 435/6.11; 435/15; 435/6.12

(57) ABSTRACT

The present invention is directed to mutations in the DGAT1 gene that produce an advantageous milk, tissue and/or growth rate profile in animals carrying the mutations. The present invention is also directed to methods of identifying animals carrying the mutations in order to facilitate the selection of animals with altered milk, tissue and/or growth rate traits.

This international patent application claims priority from New Zealand provisional patent application 573950 filed on 24 Dec. 2008, the contents of which are to be taken as incorporated herein by this reference.

### FIELD OF THE INVENTION

This invention relates to a new genetic variation associated with milk production, such as volume, and the composition of fat and protein in the milk. The new genetic variation is also associated with tissue composition of animals containing the variation. The invention also relates to the selection of animals, particularly bovine, by assaying for the presence or absence of the genetic variation.

### BACKGROUND

The genetic basis of animal fat and milk composition, particularly bovine milk production, is of immense significance to the dairy industry. An ability to modulate animal fat and milk composition and volumes has the potential to alter farming practices and to manufacture products which are tailored to meet a range of requirements. In particular, a method of genetically evaluating animals, particularly bovine, to select those which express desirable traits, such as desirable milk and meat composition, would be useful.

A genetic basis for variations in the composition of milk, for example the relative amounts of major milk proteins, and the effect of these variations on milk production characteristics and milk processing properties, has been the subject of considerable research, debate, and review. For example, international patent application PCT/NZ01/00245 (published as WO02/36824) reports that polymorphisms in the bovine diacylglycerol O-acyltransferase homolog 1 (mouse) (DGAT1) gene are associated with increased milk yield and altered milk composition, and in particular that the presence of the relatively frequent K232A polymorphism in the DGAT1 polypeptide, where the presence of an alanine amino acid at position 232 of the DGAT1 polypeptide results in a decrease in milk fat percentage, milk fat yield and milk protein percentage, while increasing milk volume and milk protein yield. The K232A polymorphism has been shown to have an effect on DGAT1 enzyme function (Grisart, B., et al., 2002, Genome Res. 12:222-231; Grisart B., et al., 2004, PNAS 101:2398-2403). Thirteen polymorphisms in the bovine DGAT1 gene are identified in Table 1 of WO 02/36824, only two of which occur in exons. The K232A polymorphism is identified as being in exon 8, and a synonymous polymorphism in exon 4, at base 5997, is also identified. DGAT1 is involved in triglyceride synthesis, and catalyses the attachment of a fatty acid onto the third position of the glycerol backbone by covalently joining a fatty acyl-CoA and a diacylglycerol. A general review of DGAT enzymes, DGAT1 and DGAT2 is provided in Yen, et al., 2008, Journal of Lipid Research, 49:2283-2301.

In another example, international patent application PCT/NZ02/00157 (published as WO2003/104492) reports that polymorphisms in the bovine growth hormone receptor (GHR) gene are associated with an increased milk volume and altered milk composition, and in particular the presence of a F279Y amino acid polymorphism results in an increased milk yield, decreased milk fat and milk protein percentage, as well as a decrease in live weight. For other characteristics of milk composition, the basis for variation is less clear.

Marker assisted selection, which provides the ability to follow a specific favourable genetic allele, involves the identification of a DNA molecular marker or markers that segregate(s) with a gene or group of genes associated with, or in part defines, a trait. DNA markers have several advantages. They are relatively easy to measure and are unambiguous, and as DNA markers are co-dominant, heterozygous and homozygous animals can be distinctively identified. Once a marker system is established, selection decisions are able to be made very easily as DNA markers can be assayed at any time after a DNA containing sample has been collected from an individual animal, whether embryonic, infant or adult.

The present invention provides a novel mutation associated with advantageous milk, tissue, colostrum and growth characteristics in animals. The present invention also provides a method for the selection of an animal, in particular a bovine, with a desired tissue composition and/or desired milk and/or colostrum production qualities such as volume, and composition of fat and protein, by direct detection of the mutation or by marker-assisted selection. The present invention also provides animals selected using the method of the invention. Furthermore, the present invention provides tissue products derived from the selected animals, such as but not limited to meat, organs, pelts, fluids, for example blood and serum, and the like, wherein said tissues typically have a decreased fat content and/or decreased degree of fat saturation. Still further, the present invention provides milk produced by, the selected animals, and dairy products produced therefrom, so as to provide the public with an alternative to the milk, dairy and tissue products currently on the market.

### SUMMARY OF THE INVENTION

This invention relates to the identification of a mutation in the DGAT1 gene. In particular, the invention relates to the identification of a mutation in a region of the DGAT1 gene equivalent to exon 16 of the bovine DGAT1 gene, the identification of several markers linked to this mutation, and the association of the mutation with the quality of milk and tissue produced by animals containing the mutation, particularly the fat composition of milk and tissue, and/or milk volume.

Accordingly, in a first aspect, the present invention provides an isolated nucleic acid molecule, the nucleic acid molecule comprising a DGAT1 nucleotide sequence encoding a DGAT1 protein, or a portion thereof, wherein the nucleic acid molecule has a mutation in a region of the DGAT1 nucleotide sequence equivalent to exon 16 of a bovine DGAT1 gene.

Reference to “a mutation” or “the mutation” is reference to any mutation in a region of the DGAT1 nucleotide sequence equivalent to exon 16 of bovine DGAT1. Hereinafter, such mutations are referred to as “a mutation of the invention” or “the mutation of the invention”.

In one embodiment, the mutation disrupts the function of the DGAT1 protein.

In further embodiments, the mutation disrupts the expression of a full-length DGAT1 protein.

In still further embodiments, the mutation disrupts the enzymatic activity of the DGAT1 protein.

In some embodiments, the mutation disrupts an exon splicing motif in the DGAT1 nucleotide sequence.

In some embodiments, the DGAT1 nucleotide sequence may encode a bovine DGAT1 protein. The bovine DGAT1 protein may be missing one or more amino acids which are encoded by exon 16 of the bovine DGAT1 gene. Alternatively, the bovine DGAT1 protein may be missing all amino acids which are encoded by exon 16 of the bovine DGAT1 gene.

In some embodiments, the mutation is a nucleotide substitution at position 8078 of the bovine DGAT1 gene as represented by GenBank Accession AY065621/GI:18642597. For example, the mutation may be an A to C nucleotide substitution at position 8078 of the bovine DGAT1 gene as represented by GenBank Accession AY065621/GI:18642597.

The present invention also provides a nucleic acid molecule according to a first aspect of the invention comprising the nucleotide sequence set forth in SEQ ID NOs: 2 or 44.

In a second aspect, the present invention provides an isolated nucleic acid molecule consisting of the nucleotide sequence set forth in SEQ ID NOs: 2 or 44.

In a third aspect, the present invention provides an isolated polypeptide, the polypeptide comprising a DGAT1 amino acid sequence, wherein the polypeptide has a mutation in a region of the DGAT1 amino acid sequence equivalent to the amino acids encoded by exon 16 of a bovine DGAT1 gene.

Reference to “a mutation” or “the mutation” in the context of this aspect of the invention is reference to any mutation in a region of the DGAT1 amino acid sequence equivalent to the amino acids encoded by exon 16 of a bovine DGAT1 gene. As indicated above, such mutations are referred to herein as “a mutation of the invention” or “the mutation of the invention”.

In one embodiment, the mutation disrupts the function of the polypeptide.

In some embodiments, the mutation disrupts the enzymatic activity of the polypeptide.

In further embodiments, the mutation disrupts the expression of a full-length DGAT1 polypeptide.

In some embodiments, the polypeptide is a bovine DGAT1 protein. For example, in one embodiment the isolated polypeptide may comprise the amino acid sequence set forth in SEQ ID NOs: 4 or 46.

In some embodiments, the bovine DGAT1 protein may be missing one or more amino acids which are encoded by exon 16 of the bovine DGAT1 gene. Alternatively, the bovine DGAT1 protein may be missing all amino acids which are encoded by exon 16 of the bovine DGAT1 gene. For example, in one embodiment the isolated polypeptide may comprise the amino acid sequence set forth in SEQ ID NOs: 47 or 48.

In a fourth aspect, the present invention provides an isolated polypeptide consisting of the amino acid sequence set forth in SEQ ID NOs: 47 or 48.

Animals carrying a mutation of the invention produce milk with an advantageous milk profile, or are capable of producing progeny that produce milk with an advantageous milk profile, wherein the advantageous milk profile is selected from one or more of a reduction in total milk fat as a percentage of whole milk, an increase in the percentage of unsaturated fatty acids in the total milk fatty acid content, a decrease in the percentage of saturated fatty acids in the total milk fatty acid content, an increase in protein yield, an increase in the percentage of omega-3 fatty acids in the total milk fatty acid content, a decreased fat hardness as indicated by a reduced solid fat content of extracted milk fat, for example at 10° C., a decrease in the ratio of milk fat:protein, an increase in the volume of milk produced, and an increase in lactose yield. Each of these characteristics is increased or decreased relative to an animal of the same breed not carrying the mutation. As used herein, “advantageous milk profile” means milk having at least one of the above-listed characteristics.

The term “fat” as used in the context of “milk fat” has the usual meaning in the art. For example, a fat refers to a triglyceride (or triacylglycerol) which contains three fatty acids attached to a glycerol backbone. In the case of milk fat, triglycerides represent up to about 98% of the total fat content, with phospholipids, cholesterol, cholesterol esters, diglycerides, monoglycerides, free fatty acids and fat-soluble vitamins making up the remainder.

The term “fatty acid” would also be well understood in the art, and refers to a carboxylic acid having an unbranched or branched hydrocarbon chain of varying length with a methyl group at one end of the chain and a carboxylic acid at the other end. A fatty acid with only single bonds in the carbon chain is known as a saturated fatty acid. In contrast, a fatty acid with a single double bond in the carbon chain is known as a monounsaturated fatty acid, and a fatty acid with two or more double bonds in the carbon chain is known as a polyunsaturated fatty acid.

In some embodiments, a bovine carrying a mutation of the invention produces milk, or is capable of producing progeny that produces milk, wherein the milk has less than about 3% total milk fat. In other embodiments, a bovine carrying a mutation of the invention produces milk, or is capable of producing progeny that produces milk, wherein the milk has at least about 27% unsaturated fatty acids in the total fatty acid content of their milk. In other embodiments, a bovine carrying a mutation of the invention produces milk, or is capable of producing progeny that produces milk, wherein the milk has less than about 57% saturated fatty acids in the total milk fatty acid content of their milk. In other embodiments, a bovine carrying a mutation of the invention produces milk, or is capable of producing progeny that produces milk, wherein the milk has at least about 1.2% of omega-3 fatty acids in the total milk fatty acid content of their milk. In other embodiments, a bovine carrying a mutation of the invention produces milk, or is capable of producing progeny that produces milk at a volume of it least 6000 litres a season under standard New Zealand farming conditions, namely dairy cattle grazing rye grass/white clover pasture.

As used in the specification; “about” means approximately or nearly, and in the context of a numerical value or range set forth herein means ±10% of the numerical value or range recited or claimed.

In a further aspect, the present invention also relates to products produced from the milk of an animal carrying a mutation of the invention, including dairy products such as creams, icecreams, yoghurts and cheeses, dairy based drinks (such as milk drinks including milk shakes, and yoghurt drinks), milk powders, and dairy based sports supplements, as well as other products including food additives such as protein sprinkles, and dietary supplement products including daily supplement tablets.

Animals carrying a mutation of the invention also typically have an advantageous tissue profile, in that they produce tissue, or are capable of producing progeny that produces tissue, having one or more qualities selected from the group consisting of a reduction in total fat as a percentage of total mass, an increase in the percentage of unsaturated fatty acids in the total fatty acid content, a decrease in the percentage of saturated fatty acids in the total fatty acid content, an increase in protein yield, an increase in the percentage of omega-3 fatty acids in the total fatty acid content, a decrease in the fat hardness as indicated by a reduced solid fat content of extracted fat, for example at 10° C., a decrease in the ratio of fat:protein, and an increase in the volume of meat produced due to a general increased growth rate of the animal. As indicated above, each of these characteristics is increased or decreased relative to an animal of the same breed not carrying the mutation. As used herein, “advantageous tissue profile” refers to a tissue of an animal having at least one of the above-listed characteristics. In effect, such animals have depressed fat synthesis, resulting in decreased fat in tissues, such as muscle, thus allowing the animals to produce leaner meat. Such animals also typically have an increased growth rate.

In a further aspect, the present invention also relates to a tissue or tissue product derived from an animal carrying a mutation of the invention. In one embodiment, the tissue or tissue product may include, but is not limited to meat, organs, pelts, fluids, for example blood and serum, and the like. These tissues typically have a decreased fat content and/or decreased degree of fat saturation.

Animals carrying a mutation of the invention also typically have an advantageous colostrum profile, in that they produce colostrum, or are capable of producing progeny that produces colostrum, having one or more qualities selected from the group consisting of a reduction in total colostrum fat as a percentage of whole colostrum, an increase in the percentage of unsaturated fatty acids in the total colostrum fatty acid content, a decrease in the percentage of saturated fatty acids in the total colostrum fatty acid content, an increase in protein yield, an increase in the percentage of omega-3 fatty acids in the total colostrum fatty acid content, a decrease in the ratio of colostrum fat:protein, and an increase in the volume of colostrum produced. Each of these characteristics is increased or decreased relative to an animal of the same breed not carrying the mutation. As used herein, “advantageous colostrum profile” means colostrum having at least one of the above-listed characteristics.

In some embodiments, animals carrying a mutation of the invention may include mammals, avian species, and aquaculture species. Mammals may include, but are not limited to, farmed mammals, such as bovine, sheep, and goats. Avian species include, but are not limited to fowl such as chickens, ducks, turkeys, and geese. Aquaculture species include, but are not limited to, fish such as salmon, trout, kingfish, barramundi, and shellfish. In one embodiment, the animal is a bovine. Such animals will generally produce meat and milk with a lower fat content compared to an animal of the same species and breed that does not carry the mutation.

There are further numerous, and separate, aspects of the invention.

In one further aspect the present invention provides a method of assessing the genetic merit of a bovine, the method including determining if the bovine comprises a nucleic acid molecule having a DGAT1 nucleotide sequence encoding a DGAT1 protein, or a portion thereof, and having a mutation in exon 16 of the DGAT1 nucleotide sequence.

In a still further aspect, the present invention provides a method of assessing the genetic merit of a bovine with respect to an advantageous milk profile, the method including determining if the bovine comprises a nucleic acid molecule having a DGAT1 nucleotide sequence encoding a DGAT1 protein, or a portion thereof, and having a mutation in exon 16 of the DGAT1 nucleotide sequence. In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In a yet further aspect, the present invention provides a method of assessing the genetic merit of a bovine with respect to an advantageous tissue profile, an advantageous colostrum profile, and/or an increased growth rate, the method including determining if the bovine comprises a nucleic acid molecule having a DGAT1 nucleotide sequence encoding a DGAT1 protein, or a portion thereof, and having a mutation in exon 16 of the DGAT1 nucleotide sequence. In one embodiment, the advantageous tissue profile relates to fat content, more preferably unsaturated fatty acid content, and particularly omega-3 fatty acid content. In a preferred embodiment the tissue is meat.

In some embodiments, the genetic merit of the bovine is identified by determining if the bovine comprises a nucleic acid molecule according to a first aspect of the invention.

In one aspect, the present invention provides a method of assessing the genetic merit of a bovine, the method including determining if the bovine comprises a polypeptide having a DGAT1 amino acid sequence with a mutation in one or more of the amino acids encoded by exon 16 of DGAT1.

In a further aspect, the present invention provides a method of assessing the genetic merit of a bovine with respect to an advantageous milk profile, the method including determining if the bovine comprises a polypeptide having a DGAT1 amino acid sequence with a mutation in one or more of the amino acids encoded by exon 16 of DGAT1. In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In a yet further aspect, the present invention provides a method of assessing the genetic merit of a bovine with respect to an advantageous tissue profile, an advantageous colostrum profile, and/or an increased growth rate, the method including determining if the bovine comprises a polypeptide having a DGAT1 amino acid sequence with a mutation in one or more of the amino acids encoded by exon 16 of DGAT1.

In some embodiments, the method includes determining if the bovine comprises a polypeptide according to a third aspect of the invention.

In some embodiments, the method includes determining the expression and/or activity of the polypeptide.

In still further embodiments, the method further comprises selecting the bovine on the basis of the identified genetic merit.

In some embodiments, the method for assessing the genetic merit of the bovine is an in vitro method.

In one aspect the present invention provides a method for selecting a bovine that produces an advantageous milk profile, or a bovine capable of producing progeny that produce an advantageous milk profile, the method including: (i) determining if the bovine comprises a nucleic acid molecule having a DGAT1 nucleotide sequence encoding a DGAT1 protein, or a portion thereof, and having a mutation in exon 16 of the DGAT1 nucleotide sequence; and (ii) selecting the bovine on the basis of the determination. In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In a further aspect the present invention provides a method for selecting a bovine that produces an advantageous tissue profile, an advantageous colostrum profile, and/or an increased growth rate, or a bovine capable of producing progeny that produce an advantageous tissue profile, an advantageous colostrum profile, and/or an increased growth rate, the method including: (i) determining if the bovine comprises a nucleic acid molecule having a DGAT1 nucleotide sequence encoding a DGAT1 protein, or a portion thereof, and having a mutation in exon 16 of the DGAT1 nucleotide sequence; and (ii) selecting the bovine on the basis of the determination. Preferably, the tissue is meat.

In some embodiments, the selection method includes determining if the bovine comprises a nucleic acid molecule according to a first aspect of the invention.

In one aspect the present invention provides a method for selecting a bovine that produces an advantageous milk profile, or a bovine capable of producing progeny that produce an advantageous milk profile, the method including: (i) determining if the bovine comprises a polypeptide having a DGAT1 amino acid sequence with a mutation in one or more of the amino acids encoded by exon 16 of DGAT1; and (ii) selecting the bovine on the basis of the determination. In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In a further aspect, the present invention provides a method for selecting a bovine that produces an advantageous tissue profile, an advantageous colostrum profile, and/or an increased growth rate, or a bovine capable of producing progeny that produce an advantageous, tissue profile, an advantageous colostrum profile, and/or an increased growth rate, the method including: (i) determining if the bovine comprises a polypeptide having a DGAT1 amino acid sequence with a mutation in one or more of the amino acids encoded by exon 16 of DGAT1; and (ii) selecting the bovine on the basis of the determination.

In some embodiments, the selection method includes determining if the bovine comprises a polypeptide according to a third aspect of the invention.

In some embodiments, the selection method includes determining the expression and/or activity of the polypeptide. In one embodiment, the expression of the polypeptide is determined from RNA encoding the polypeptide. The RNA may be transcribed from a nucleic acid molecule according to a first aspect of the invention.

In one embodiment, the expression and/or activity of the polypeptide is determined by measuring an amount of the polypeptide, absence of the polypeptide, and/or amount of the polypeptide compared to an amount of wild-type DGAT1 polypeptide expressed by the bovine.

In some embodiments, the method for selecting the bovine is an in vitro method.

In one aspect, the present invention provides a method of assessing the genetic merit of a bovine with respect to an advantageous milk profile, the method including determining a DGAT1 exon 16 allelic profile of said bovine.

In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In a further aspect, the present invention provides a method of assessing the genetic merit of a bovine with respect to an advantageous tissue profile, an advantageous colostrum, profile, and/or an increased growth rate, the method including determining a DGAT1 exon 16 allelic profile of said bovine.

In one embodiment, the DGAT1 exon 16 allelic profile is determined from nucleic acid molecules obtained from said bovine. In one embodiment, the nucleic acid molecules are DNA. In another embodiment, the nucleic acid molecules are RNA, such as mRNA or hnRNA. In some embodiments, the method includes determining the presence or absence of a nucleic acid molecule according to a first aspect of the invention.

In one embodiment, the DGAT1 exon 16 allelic profile is determined from polypeptide obtained from said bovine. In one embodiment, the method includes determining the presence or absence of a DGAT1 polypeptide according to a third aspect of the invention.

In one embodiment, the DGAT1 exon 16 allelic profile is determined using a polymorphism in linkage or in linkage disequilibrium with a DGAT1 exon 16 allele. In one embodiment, the polymorphism in linkage or linkage disequilibrium with the DGAT1 exon 16 allele are on chromosome 14 and are selected from the group consisting of ARS-BFGL-NGS-4939, Hapmap52798-ss46526455, Hapmap29758-BTC-003619, BFGL-NGS-18858, Hapmap24717-BTC-002824, and Hapmap24718-BTC-002945.

In a further embodiment, the method further includes determining an allelic profile of said bovine at one or more additional genetic loci associated with an advantageous milk profile. For example in one embodiment, the genetic loci are one or more polymorphisms in one or more genes associated with milk volume and/or content. In one embodiment, the one or more polymorphisms in one or more genes are associated with fat metabolism. In a further embodiment, the DGAT1 exon 16 allelic profile and the allelic profile at one or more additional genetic loci act synergistically to produce an advantageous milk profile. In a still further embodiment, the one or more additional genetic loci are on the same chromosome as DGAT1. For example, the polymorphism may encode a lysine to alanine substitution at amino acid position 232 of the bovine DGAT1 protein. Alternatively, the one or more additional genetic loci are located on a different chromosome to DGAT1.

In another aspect, the present invention provides a method for assessing the genetic merit of a bovine with respect to an advantageous milk profile, the method including determining if the bovine comprises a nucleic acid molecule encoding: (i) a polypeptide (A) having biological activity of wild-type DGAT1; or (ii) a polypeptide (B) having a DGAT1 amino acid sequence with a mutation in one or more of the amino acids encoded by exon 16 of DGAT1; or (iii) polypeptide A and polypeptide B, wherein absence of a nucleic acid molecule encoding polypeptide A and presence of a nucleic acid molecule encoding polypeptide B, or presence of both a nucleic acid molecule encoding polypeptide A and a nucleic acid molecule encoding polypeptide B, indicates an advantageous milk profile.

As used herein, the term “wild-type DGAT1” refers to a DGAT1 nucleic acid molecule or DGAT1 polypeptide which does not comprise a mutation of the invention. For example, in one embodiment, a nucleic acid, molecule encoding a polypeptide having biological activity of wild-type DGAT1 will have an adenine nucleotide at position 8078 of the bovine DGAT1 gene as represented by GenBank Accession AY065621/GI:18642597. Such a nucleic acid molecule may have the nucleotide sequence set forth in SEQ ID NOs: 1 or 43. Accordingly, the encoded polypeptide, which has the biological activity of wild-type DGAT1 will have a methionine at amino acid position 435 of the bovine DGAT1 protein as represented by GenBank Accession AAL49962/GI:18642598. Such a polypeptide may have the amino acid sequence set forth in SEQ ID NOs: 3 or 45.

In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In another aspect, the present invention provides a method for assessing the genetic merit of a bovine with respect to an advantageous tissue profile, an advantageous colostrum profile, and/or an increased growth rate, the method including determining if the bovine comprises a nucleic acid molecule encoding: (i) a polypeptide (A) having biological activity of wild-type DGAT1; or (ii) a polypeptide (B) having a DGAT1 amino acid sequence with a mutation in one or more of the amino acids encoded by exon 16 of DGAT1; or (iii) polypeptide A and polypeptide B, wherein absence of a nucleic acid molecule encoding polypeptide A and presence of a nucleic acid molecule encoding polypeptide B, or presence of both a nucleic acid molecule encoding polypeptide A and a nucleic acid molecule encoding polypeptide B, indicates an advantageous tissue profile, an advantageous colostrum profile, and/or an increased growth rate.

In one embodiment, the nucleic acid molecule is DNA or RNA. The DNA may be, or the RNA may be transcribed from, a nucleic acid molecule according to a first aspect of the invention. In one embodiment, the method for assessing the genetic merit further comprises ascertaining the amount of RNA encoding polypeptide B.

In one embodiment, polypeptide A may comprise the amino acid sequence set forth in SEQ ID NO: 3 or 45.

In one embodiment, polypeptide B is a polypeptide according to a third aspect of the invention.

In another aspect, the present invention provides a method for assessing the genetic merit of bovine with respect to an advantageous milk profile, the method including determining if the bovine comprises: (i) a polypeptide (A) having biological activity of wild-type DGAT1; or (ii) a polypeptide (B) having a DGAT1 amino acid sequence with a mutation in one or more of the amino acids encoded by exon 16 of DGAT1; or (iii) polypeptide A and, polypeptide B, wherein absence of polypeptide A and presence of polypeptide B, or presence of both polypeptide A and polypeptide B, indicates an advantageous milk profile.

In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In another aspect, the present invention provides a method for assessing the genetic merit of bovine with respect to an advantageous tissue profile, an advantageous colostrum profile, and/or an increased growth rate, the method including determining if the bovine comprises: (i) a polypeptide (A) having biological activity of wild-type DGAT1; or (ii) a polypeptide (B) having a DGAT1 amino acid sequence with a mutation in one or more of the amino acids encoded by exon 16 of DGAT1; or (iii) polypeptide A and polypeptide B, wherein absence of polypeptide A and presence of polypeptide B, or presence of both polypeptide A and polypeptide B, indicates an advantageous tissue profile, an advantageous colostrum profile, and/or an increased growth rate.

In one embodiment, the method further comprises ascertaining the amount and/or activity of polypeptide B.

In one embodiment, polypeptide A may comprise the amino acid sequence set forth in SEQ ID NO: 3 or 45.

In one embodiment, polypeptide B is a polypeptide according to a third aspect of the invention.

In another aspect, the present invention provides a method for determining the DGAT1 genotype of a bovine, the method including determining if a nucleic acid molecule obtained from the bovine is: (i) a nucleic acid molecule (A) encoding a polypeptide having biological activity of wild-type DGAT1; or (ii) a nucleic acid molecule (B) having a DGAT1 nucleotide sequence encoding a DGAT1 protein, and having a mutation in exon 16 of the DGAT1 nucleotide sequence, wherein the nucleic acid molecule obtained from the bovine is uncontaminated by heterologous nucleic acid.

In one embodiment, nucleic acid molecule B encodes a polypeptide according to a third aspect of the invention.

In one embodiment, nucleic acid molecule A may comprise the nucleotide sequence set forth in SEQ ID NO: 1 or 43.

In one embodiment, nucleic acid molecule B is a nucleic acid molecule according to a first aspect of the invention.

In another aspect, the present invention provides a method for determining the DGAT1 genotype of a bovine, the method including determining if a polypeptide obtained from the bovine is: (i) a polypeptide (A) having biological activity of wild-type DGAT1; or (ii) a polypeptide (B) having a DGAT1 amino acid sequence with a mutation in one or more of the amino acids encoded by exon 16 of DGAT1, wherein the polypeptide obtained from the bovine is uncontaminated by heterologous polypeptide.

In one embodiment, the method includes determining the expression and/or activity of the polypeptide.

In another embodiment, the method includes mass spectrometric analysis of the polypeptide or peptides derived from the polypeptide.

In one embodiment, polypeptide B is a polypeptide according to a third aspect of the invention.

Although some of the methods described above are referred to in the context of a bovine, it will be appreciated that they may equally apply to other animals, such as, but not limited to those described above.

In a further aspect, the present invention includes a probe comprising a nucleic acid molecule, wherein said probe hybridises under stringent conditions to a nucleic acid molecule according to a first aspect of the invention. The present invention is also directed to a diagnostic kit containing the probe.

The present invention also includes a primer composition for detection of a nucleic acid molecule according to a first aspect of the invention. In one embodiment, the primer composition includes one or more nucleic acid molecules substantially complementary to a portion of the nucleic acid molecule according to a first aspect of the invention or its complement. For example, the primer composition may include nucleic acid molecules having the nucleotide sequences set forth in SEQ ID NOs: 5, 6 and 7. Diagnostic kits including such a primer composition are also envisaged.

The present invention further includes an antibody composition for detection of a polypeptide according to a third aspect of the invention. Diagnostic kits including such an antibody composition, together with instructions for use, are also envisaged.

The present invention further provides a diagnostic kit for detecting a nucleic acid molecule according to a first aspect of the invention, the kit including first and second primers for amplifying the nucleic acid molecule, or a portion thereof, the primers being complementary to nucleotides of the nucleic acid molecule which are upstream and downstream, respectively, to the mutation.

In one embodiment, at least one of the primers of the diagnostic kit includes nucleotides which are complementary to a non-coding region of the nucleic acid molecule. The diagnostic kit may also include a third primer complementary to the mutation.

In one embodiment, the nucleic acid molecule to which the primers bind encodes a polypeptide according to a third aspect of the invention.

In a further aspect, the present invention provides a method of assessing the genetic merit of a bovine with respect to an advantageous milk profile, the method including determining the presence or absence of an A nucleotide at position 8078 of the bovine DGAT1 gene as represented by GenBank Accession AY065621/GI:18642597.

In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In a further aspect the present invention provides a method of assessing the genetic merit of a bovine with respect to an advantageous tissue profile, an advantageous colostrum profile, and/or an increased growth rate, the method including determining the presence or absence of an A nucleotide at position 8078 of the bovine DGAT1 gene as represented by GenBank Accession AY065621/GI:18642597.

In a further aspect, the present invention provides a method of assessing the genetic merit of a bovine with respect to an advantageous milk profile, the method including determining the presence or absence of a C nucleotide at position 8078 of the bovine DGAT1 gene as represented by GenBank Accession AY065621/GI:18642597.

In one embodiment, the advantageous milk profile is selected from one or more of a reduction in total milk fat content, an increase in the percentage of unsaturated fatty acids in the total milk fatty acid, content, an increase in the percentage of omega-3 fatty acids in the total milk fatty acid content, a decrease in the ratio of milk fat:protein, and an increase in the volume of milk produced.

In a further aspect, the present invention provides a method of assessing the genetic merit of a bovine with respect to an advantageous tissue profile, an advantageous colostrum profile, and/or an increased growth rate, the method including determining the presence or absence of a C nucleotide at position 8078 of the bovine DGAT1 gene as represented by GenBank Accession AY065621/GI:18642597.

In a further aspect, the present invention provides a method of assessing the genetic merit of a bovine with respect to an advantageous milk profile, the method including determining the presence or absence of a CC genotype at position 8078 of the bovine DGAT1 gene as represented by GenBank Accession AY065621/GI:18642597.

In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In a further aspect, the present invention provides a method of assessing the genetic merit of a bovine with respect to an advantageous tissue profile, an advantageous colostrum profile, and/or an increased growth rate, the method including determining the presence or absence of a CC genotype at position 8078 of the bovine DGAT1 gene as represented by GenBank Accession AY065621/GI:18642597.

In a further aspect, the present invention provides a method of assessing the genetic merit of a bovine with respect to an advantageous milk profile, the method including determining the presence of an AC genotype at position 8078 of the bovine DGAT1 gene as represented by GenBank Accession AY065621/GI:18642597.

In one embodiment, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In a further aspect, the present invention provides a method of assessing the genetic merit of a bovine with respect to an advantageous tissue profile, an advantageous colostrum profile, and/or an increased growth rate, the method including determining the presence of an AC genotype at position 8078 of the bovine DGAT1 gene as represented by GenBank Accession AY065621/GI:18642597.

In another aspect, the present invention provides a method for selecting a bovine with a genotype indicative of an advantageous milk profile, the method including: (i) determining a DGAT1 exon 16 allelic profile of said bovine as referred to in the aforementioned aspects; and (ii) selecting the bovine on the basis of the determination. For example, the advantageous milk profile may include one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced.

In a further aspect, the present invention provides a method for selecting a bovine with a genotype indicative of an advantageous tissue profile, an advantageous colostrum profile, and/or an increased growth rate, the method including: (i) determining a DGAT1 exon 16 allelic profile of said bovine as referred to in the aforementioned aspects; and (ii) selecting the bovine on the basis of the determination.

In a further aspect, the present invention provides a method for selecting a bovine with a DGAT1 exon 16 allelic profile indicative of an advantageous milk profile, the method including: (i) determining the absence of a CC genotype at position 8078 of the bovine DGAT1 gene as represented by GenBank Accession AY065621/GI:18642597; and selecting the bovine on the basis of the determination.

In a further aspect, the present invention provides a method for selecting a bovine with a DGAT1 exon 16 allelic profile indicative of an advantageous tissue profile, an advantageous colostrum profile, and/or an increased growth rate, the method including: (i) determining the absence of a CC genotype at, position 8078 of the bovine DGAT1 gene as represented by GenBank Accession AY065621/GI:18642597; and selecting the bovine on the basis of the determination.

In a further aspect, the present invention provides a method for selecting a bovine with a DGAT1 exon 16 allelic profile indicative of an advantageous milk profile, the method including: (i) determining the absence of an A nucleotide at position 8078 of the bovine DGAT1 gene as represented by GenBank Accession AY065621/GI:18642597; and (ii) selecting the bovine on the basis of the determination.

In a further aspect, the present invention provides a method for selecting a bovine with a DGAT1 exon 16 allelic profile indicative of an advantageous tissue profile, an advantageous colostrum profile, and/or an increased growth rate, the method including: (i) determining the absence of an A nucleotide at position 8078 of the bovine DGAT1 gene as represented by GenBank Accession AY065621/GI:18642597; and (ii) selecting the bovine on the basis of the determination.

In a further aspect, the present invention provides a method for selecting a bovine with a DGAT1 exon 16 allelic profile indicative of an advantageous milk profile, the method including: (i) determining the presence of a C nucleotide at position 8078 of the bovine DGAT1 gene as represented by GenBank Accession AY065621/GI:18642597; and (ii) selecting the bovine on the basis of the determination.

In a further aspect, the present invention provides a method for selecting a bovine with a DGAT1 exon 16 allelic profile indicative of an advantageous tissue profile, an advantageous colostrum profile, and/or an increased growth rate, the method including: (i) determining the presence of a C nucleotide at position 8078 of the bovine DGAT1 gene as represented by GenBank Accession AY065621/GI:18642597; and (ii) selecting the bovine on the basis of the determination.

In a further aspect, the present invention provides a method for selecting a bovine with a DGAT1 exon 16 allelic profile indicative of an advantageous milk profile, the method including: (i) determining the presence of a CC genotype at position 8078 of the bovine DGAT1 gene as represented by GenBank Accession AY065621/GI:18642597; and (ii) selecting the bovine on the basis of the determination.

In a further aspect, the present invention provides a method for selecting a bovine with a DGAT1 exon 16 allelic profile indicative of an advantageous tissue profile, an advantageous colostrum profile, and/or an increased growth rate, the method including: (i) determining the presence of a CC genotype at position 8078 of the bovine DGAT1 gene as represented by GenBank Accession AY065621/GI:18642597; and (ii) selecting the bovine on the basis of the determination.

In a further aspect the present invention provides a method for selecting a bovine with a DGAT1 exon 16 allelic profile indicative of an advantageous milk profile, the method including: (i) determining the presence of an AC genotype at position 8078 of the bovine DGAT1 gene as represented by GenBank Accession AY065621/GI:18642597; and (ii) selecting the bovine on the basis of the determination.

In a further aspect, the present invention provides a method for selecting a bovine with a DGAT1 exon 16 allelic profile indicative of an advantageous tissue profile, an advantageous colostrum profile, and/or an increased growth rate, the method, including: (i) determining the presence of an AC genotype at position 8078 of the bovine DGAT1 gene as represented by GenBank Accession AY065621/GI:18642597; and (ii) selecting the bovine on the basis of the determination.

In some embodiments, the advantageous milk profile is selected from one or more of a reduction in total milk fat content, an increase in the percentage of unsaturated fatty acids in the total milk fatty acid content, an increase in the percentage of omega-3 fatty acids in the total milk fatty acid content, a decrease in the ratio of milk fat:protein, and an increase in the volume of milk produced

In some embodiments, the presence of the C nucleotide, CC genotype or AC genotype is ascertained from genomic DNA or RNA which has been obtained from the bovine, or from cDNA produced from the RNA.

In some embodiments, the presence of the C nucleotide, CC genotype or AC genotype is ascertained by detecting the presence of a codon encoding leucine at amino acid position 435 of the bovine DGAT1 protein as represented by GenBank Accession AAL49962/GI:18642598.

In some embodiments, the presence of the AC genotype is ascertained by detecting the presence of a codon encoding methionine at amino acid position 435 of the bovine DGAT1 protein as represented by GenBank Accession AAL49962/GI:18642598.

In some embodiments, the presence of the C nucleotide, CC genotype or AC genotype is ascertained by sequencing a DGAT1 nucleic acid molecule obtained from the bovine.

In further embodiments, the determination comprises the step of amplifying a DGAT1 nucleic acid molecule from genomic DNA or RNA which has been obtained from the bovine, or from cDNA produced from the RNA.

In one embodiment, the amplifying is performed by PCR.

In one embodiment, amplification is by use of primers which include nucleic acid molecules having at least about 10 contiguous nucleotides of, or complementary to, the nucleotide, sequence set, forth in one of SEQ ID NOs:1, 2, 43 and 44 or a naturally occurring flanking sequence.

In one embodiment at least one, of the primers includes a nucleic acid molecule having a nucleotide sequence set forth in one of SEQ ID NOs: 5, 6 and 7.

In some embodiments; the determination comprises the step of restriction enzyme digestion of a DGAT1 nucleic acid molecule derived from the bovine. Such digestion may also be performed on a product of the PCR amplification described above.

In some embodiments, the presence of the C nucleotide, CC genotype or AC genotype is ascertained by mass spectrometric analysis of a DGAT1 nucleic acid molecule derived from the bovine.

In some embodiments, the presence of the C nucleotide, CC genotype or AC genotype is ascertained by hybridisation of one or more probes, the one or more probes comprising a nucleic acid molecule having a nucleotide sequence of, or complementary to, a portion of the nucleotide sequence set forth in one of SEQ ID NOs:1, 2, 43 and 44.

In one embodiment, the one or more probes comprise a nucleic acid molecule having at least about 10 or more contiguous nucleotides of, or complementary to, the nucleotide sequence set forth in one of SEQ ID NOs:1, 2, 43 and 44.

In some embodiments, the one or more probes comprise a nucleic acid molecule having an A nucleotide or a C nucleotide corresponding to position 8078 of the bovine DGAT1 gene as represented by GenBank Accession AY065621/GI:18642597.

In some embodiments, the presence of the C nucleotide, CC genotype or AC genotype is ascertained by analysis of a DGAT1 polypeptide obtained from the bovine.

In one embodiment, the presence of the AC genotype is ascertained by detecting the presence of methionine at amino acid position 435 of the bovine DGAT1 protein as represented by GenBank Accession AAL49962/GI:18642598.

In a further embodiment, the presence of the AC genotype is ascertained by detecting the presence of leucine at amino acid position 435 of the bovine DGAT1 protein as represented by GenBank Accession AAL49962/GI:18642598.

In still a further aspect the present invention provides a method of selecting a herd of bovine, the method including: (i) selecting a plurality of bovine using a method according to the aforementioned aspects of the invention; and (ii) segregating and collecting the selected bovine to form the herd. In one embodiment, the invention further provides a herd of bovine, selected by such a method.

In a further aspect the present invention provides a genetically modified animal which may include a transgenic non-human animal comprising a nucleic acid molecule comprising a DGAT1 nucleotide sequence encoding a DGAT1 protein, or portion thereof, wherein the nucleic acid molecule has a mutation in a region of the DGAT1 nucleotide sequence equivalent to exon 16 of a bovine DGAT1 gene.

In one embodiment, the transgenic non-human animal comprises a nucleic acid molecule according to a first aspect of the invention. The invention also provides a clone produced from the non-human animal. Techniques to create a transgenic non-human animal and techniques for cloning and multiplication of transgenic animals are known in the art, and are described in further detail below.

In a further aspect, the present invention provides a transgenic bovine comprising a nucleic acid molecule comprising a DGAT1 nucleotide sequence encoding a DGAT1 protein, or portion thereof, wherein the nucleic acid molecule has a mutation in a region of the DGAT1 nucleotide sequence equivalent to exon 16 of a bovine DGAT1 gene. In one embodiment, the transgenic bovine comprises a nucleic acid molecule according to a first aspect of the invention.

In one embodiment, the present invention provides a clone produced from a transgenic animal or transgenic bovine described above.

In a further aspect, the present invention provides a bovine or transgenic bovine selected by the aforementioned selection methods referred to above. In one embodiment, the present invention provides a clone produced from such bovine.

In some embodiments, the transgenic non-human animal, transgenic bovine or selected bovine produces milk, or is capable of producing progeny that produces milk, having one or more qualities, selected from the group consisting of a reduction in total milk fat as a percentage of whole milk, an increase in the percentage of unsaturated fatty acids in the total milk fatty acid content, a decrease in the percentage of saturated fatty acids in the total milk fatty acid content, an increase in protein yield, an increase in the percentage of omega-3 fatty acids in the total milk fatty acid content, a decreased fat hardness as indicated by a reduced solid fat content of extracted milk fat, for example at 10° C., a decrease in the ratio of milk fat:protein, an increase in volume of milk produced, and an increase in lactose yield, when compared to an animal or bovine of the same breed not carrying the mutation.

In one embodiment, the bovine or transgenic bovine produces at least 6000 litres of milk in a season under standard New Zealand farming conditions, namely dairy cattle grazing rye grass/white clover pasture. In some embodiments, the bovine or transgenic bovine produces milk with less than about 3% total milk fat. In some embodiments, the bovine or transgenic bovine produces milk with at least about 27% unsaturated fatty acids in the total milk fatty acid content. In some embodiments, the bovine or transgenic bovine produces milk with less than about 57% saturated fatty acids in the total milk fatty acid content. In some embodiments, the bovine or transgenic bovine produces milk with at least about 1.2% of omega-3 fatty acids in the total milk fatty acid content.

In a further aspect the present invention provides milk produced by the aforementioned transgenic non-human animal, transgenic bovine or bovine.

In a still further aspect the present invention provides a product produced from the aforementioned milk. For example, the product may be selected from the group consisting of ice cream, yoghurt, cheese, a dairy based drink, a milk drink, a milk shake, a yoghurt drink, a milk powder, a dairy based sports supplement, a food additive, a protein sprinkle, a dietary supplement product and a daily supplement tablet.

In a yet further aspect the present invention provides semen or eggs produced by a transgenic non-human animal, transgenic bovine or bovine according to the aforementioned aspects of the invention.

In some embodiments, the transgenic non-human animal, transgenic bovine or selected bovine produces tissue, or is capable of producing progeny that produces tissue, having one or more qualities selected from the group consisting of a reduction in total fat as a percentage of total mass, an increase in the percentage of unsaturated fatty acids in the total fatty acid content, a decrease in the percentage of saturated fatty acids in the total fatty acid content, an increase in protein yield, an increase in the percentage of omega-3 fatty acids in the total fatty acid content, a decrease in fat hardness as indicated by a reduced solid fat content of extracted fat, for example at 10° C., a decrease in the ratio of fat:protein, and an increase in volume of meat produced due to a general increased growth rate of the animal, when compared to an animal or bovine of the same breed not carrying the mutation.

In a further aspect the present invention provides a tissue or tissue product derived from the transgenic non-human animal, transgenic bovine or bovine referred to above. In one embodiment, the tissue or tissue product is selected from the group consisting of meat, an organ, a pelt, blood and serum.

In some embodiments, the transgenic non-human animal, transgenic bovine or selected bovine produces colostrum, or is capable of producing progeny that produces colostrum, having one or more qualities selected from the group consisting of a reduction in, total colostrum fat as a percentage of whole colostrum, an increase in the percentage of unsaturated fatty acids in the total colostrum fatty acid content, a decrease in the percentage of saturated fatty acids in the total colostrum fatty acid content, an increase in protein yield, an increase in the percentage of omega-3 fatty acids in the total colostrum fatty acid content, a decrease in the ratio of colostrum fat:protein, and an increase in the volume of colostrum produced.

In a further aspect the present invention provides, a use of a nucleic acid molecule comprising a DGAT1 nucleotide sequence encoding a DGAT1 protein, or portion thereof, to produce a transgenic non-human animal, wherein the nucleic acid molecule has a mutation in a region of the DGAT1 nucleotide sequence equivalent to exon 16 of a bovine DGAT1 gene. In one embodiment, the nucleic acid molecule is a nucleic acid molecule according to a first aspect of the invention.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### DETAILED DESCRIPTION OF THE INVENTION

The present invention is predicated in part on the identification for the first time of a mutation in the diacylglycerol O-acyltransferase homolog 1 (mouse) (DGAT1) gene in a region equivalent to exon 16 of a bovine DGAT1 gene. Accordingly, the present invention provides an isolated nucleic acid molecule, the nucleic acid molecule comprising a DGAT1 nucleotide sequence encoding a DGAT1 protein, or a portion thereof, wherein the nucleic acid molecule has a mutation in a region of the DGAT1 nucleotide sequence equivalent to exon 16 of a bovine DGAT1 gene.

The isolated nucleic acid molecule may be genomic DNA comprising all or part of the DGAT1 nucleotide sequence. For example, the nucleic acid molecule may be genomic DNA comprising the entire coding and non-coding regions of the DGAT1 nucleotide sequence, with, or without associated 5′ and 3′ untranslated regions. Alternatively, the nucleic acid molecule may be genomic DNA comprising only the portion of the DGAT1 nucleotide sequence containing exon 16, or the equivalent thereof, with or without flanking intronic sequences.

The isolated nucleic acid molecule may also be RNA, for example mRNA or hnRNA, which encodes all, or a portion, of the DGAT1 protein. As would be understood by a skilled addressee, the nucleic acid molecule may also include cDNA produced from the mRNA. Standard techniques known in the art, and which are generally described in Sambrook J et al., (2001), Molecular cloning: a laboratory manual. Third Edition. (Cold Spring Harbour Laboratory Press, New York), may be used to produce cDNA from mRNA. This typically involves reverse transcribing from a nucleic acid sample containing DGAT1 mRNA with subsequent PCR amplification of the reverse transcribed product.

In some embodiments, a mutation of the invention disrupts the function and/or expression of the DGAT1 protein. With respect to function, by “disrupt” is meant that the level of function of the protein is reduced or eliminated when compared to the level of function of wild-type DGAT1 protein. With respect to expression, by “disrupt” is meant that the level of expression of the full-length mutated protein is reduced or eliminated when compared to the level of expression of wild-type DGAT1 protein. In one embodiment, the mutation disrupts the enzymatic activity of the DGAT1 protein.

A disruption in the function, expression and/or activity of the DGAT1 protein can be measured in a number of ways as would be known in the art. For example, DGAT1 encodes an enzyme which catalyses the reaction in which diacylglycerol is covalently joined to fatty acyl-CoAs to form triglycerides as major constituents of fat. Therefore, a disruption to the enzymatic activity of the DGAT1 protein may be determined by measuring the extent to which incorporation of oleoyl-CoA (a fatty acyl-CoA) into triglyceride is reduced or eliminated.

A mutation of the invention may include a nucleotide substitution, nucleotide deletion, nucleotide insertion, or any other mutation in exon 16 which alters the nucleotide sequence from that of the wild-type DGAT1 sequence, as set forth in SEQ ID NOs: 1 or 43. In one embodiment, the mutation is a nucleotide substitution which disrupts an exon splicing motif present in exon 16 of DGAT1. For example, the inventors have found an adenine (A) to cytosine (C) nucleotide substitution at position 8078 of the bovine DGAT1 gene, as represented by GenBank Accession AY065621/GI:18642597, incorporated herein by reference. This is equivalent to nucleotide position 1303 of the coding sequence of the wild-type bovine DGAT1 gene. The position of this mutation will, herein be referenced with respect to the nucleotide sequence of GenBank Accession AY065621/GI:18642597. Therefore, this mutation will be referred to herein as “A8078C” or “8078C”.

The A8078C nucleotide substitution occurs in a portion of the DGAT1 nucleotide sequence that defines a putative exon splicing motif (ATGATG) which is predicted, to enhance splicing of exon 16 during transcription. Substitution of the A nucleotide at position 8078 with a C nucleotide alters the nucleotide composition of the splicing motif to CTGATG, thereby disrupting the predicted splicing enhancer function. In effect, exon 16 is spliced out during transcription such that it is not incorporated into a DGAT1, mRNA nucleic acid molecule. Accordingly, a DGAT1 polypeptide is translated which is missing the 21 amino acids encoded by exon 16.

It would be expected and understood by a person skilled in the art that other mutations in exon 16 (including mutations in the exon splicing motif), which disrupt the function, activity and/or expression of the encoded DGAT1 polypeptide are also encompassed by the present invention. Furthermore, other mutations occurring outside of exon 16, but which result in the deletion of one or more amino acids of exon 16, and which disrupt the function, activity and/or expression of the encoded DGAT1 polypeptide, are also encompassed by the present invention. Therefore, the invention is not limited to the A8078C nucleotide mutation as exemplified herein. The A8078C nucleotide mutation merely demonstrates the importance of the amino acids encoded by exon 16 for DGAT1 function.

Accordingly, the present invention encompasses an isolated nucleic acid molecule encoding a DGAT1 polypeptide which is missing one or more amino acids which are encoded by exon 16 of the DGAT1 gene. With reference to the A8078C nucleotide mutation, the isolated nucleic acid encodes a DGAT1 polypeptide missing all 21 amino acids encoded by exon 16. The nucleotide sequence of such a nucleic acid molecule is set forth in SEQ ID NOs: 2 or 44.

Where exon 16 is not excised during RNA processing, a nucleic acid molecule having the A8078C nucleotide mutation encodes a DGAT1 polypeptide having a methionine to leucine amino acid substitution at position 435 of DGAT1.

The A8078C mutation was originally detected in a bovine, and subsequently her pedigree, which are of the Holstein-Friesian breed. It will be clear to a person skilled in the art that this mutation, or any other mutation of the invention, is not intended to be limited to the Holstein-Friesian breed, or indeed bovine species in general. A mutation of the invention may, be introduced into other animals or even cattle of different breeds by way of cross-breeding techniques, or other methods, such as transgenics, as described further below; A mutation of the invention may therefore be useful, for example, in other milk breeds such as Jersey, Guernsey, Brown Swiss, Milking Shorthorn and many others. A mutation of the invention may also be useful in dual purpose (for example Brown Swiss) and beef breeds such as Angus and Hereford given as examples of Bos taurus species, and Brahman and Zebu as examples of Bos indicus species, and any other species of the Bos genus used in animal production, as would be known in the art.

The present invention also provides an isolated polypeptide comprising a DGAT1 amino acid sequence, wherein the polypeptide has a mutation in a region of the DGAT1 amino acid sequence equivalent to the amino acids encoded by exon 16 of a DGAT1 gene. The mutation in the DGAT1 polypeptide disrupts the function, expression and/or enzymatic activity of the polypeptide. The term “disrupt” in this context has the same meaning as described above, The mutation may be an amino acid substitution, deletion, insertion or any other mutation in the amino acids encoded by exon 16 which alters the amino acid sequence from that of the wild-type DGAT1 sequence, as set forth in SEQ ID NOs: 3 or 45. In one embodiment, the mutation is in the codon encoding methionine at position 435 of the bovine DGAT1 protein as represented by GenBank Accession AAL49962/GI:18642598, incorporated herein by reference. It would be expected that the A8078C nucleotide mutation in this codon would give rise to a methionine (M) to leucine (L) amino acid substitution at position 435 of the DGAT1 polypeptide, thereby giving rise to the polypeptide comprising the amino acid sequence set forth in SEQ ID NOs: 4 or 46. Such a mutation will be referred to herein as M435L. However, as indicated above, because the A8078C nucleotide mutation disrupts a putative exon splicing motif, and therefore in the majority of instances exon 16 is spliced out during RNA processing, all 21 amino acids encoded by exon 16 are deleted in the corresponding DGAT1 polypeptide. Accordingly, in one embodiment of the present invention, the isolated polypeptide comprises the amino acid sequence set forth in SEQ ID NOs: 47 or 48. Such a mutation will be referred to herein as Δ418-438 or Δ16.

Methods for the isolation of the nucleic acid molecules and polypeptides of the invention are well known, and are generally, described in Sambrook J et al., 2001 (supra). The nucleic acid molecules and polypeptides of the invention are typically isolated from samples taken from animals. For example, such, may be taken from milk, tissues, blood, serum, plasma, cerebrospinal fluid, urine, semen, hair or saliva of the animal. Tissue samples may be obtained using standard techniques such as cell scrapings or biopsy techniques.

Polymorphisms in the bovine DGAT1 gene have previously been associated with increased milk yield and altered milk composition, and in particular the presence of a K232A amino acid substitution in DGAT1 resulting from a polymorphism in the DGAT1 gene. The polymorphism is an AA to GC di-nucleotide substitution at nucleotide positions 694 and 695 of the DGAT1 coding sequence. Bovine containing this polymorphism produce milk with a decreased milk fat percentage, milk fat yield and milk protein percentage, and an increased milk volume and milk protein yield (see WO02/36824).

DGAT1 is involved in triglyceride synthesis, and variation of triglyceride synthesis in a bovine is expected to affect triglyceride levels in milk and also fat content and/or composition of tissue (Wang J Y et al., 2007, Lipids in Health and Disease 6:2-10; White S N et al., 2007, J. Anim. Sci. 85:1-10).

A mutation of the present invention has been found to be associated with an advantageous milk profile and/or an advantageous tissue profile and/or an increased growth rate in animals containing the mutation. As used herein, the word “animal” includes mammals, avian species, and aquaculture species. Mammals may include, but are not limited to, farmed mammals, such as bovine, sheep, and goats. Avian species include, but are not limited to fowl such as chickens, ducks, turkeys, and geese. Aquaculture species include, but are not limited to, fish such as salmon, trout, kingfish, barramundi, and shellfish. In a preferred embodiment, the animal is a bovine.

As used herein, an “advantageous milk profile” refers to milk obtained from the animal which has at least one or more qualities selected from the group consisting of a reduction in total milk fat as a percentage of whole milk, an increase in the percentage of unsaturated fatty acids in the total milk fatty acid content, an increase in protein yield, a decrease in the percentage of saturated fatty acids in the total milk fatty acid content, an increase in the percentage of, omega-3 fatty acids in the total milk fatty acid content, a decrease in fat hardness as indicated by a reduced solid fat content of extracted milk fat, for example at 10° C., a decrease in the ratio of milk fat:protein, an increase in the volume of milk produced, an increase in colostrum yield, and an increase in lactose yield. Each of these qualities is increased or decreased relative to an animal of the same breed not carrying the mutation of the invention.

A mutation of the invention may affect the level of free fatty acids, for example in milk, by disrupting the function of the wild-type DGAT1 to catalyze the attachment of fatty acids to the glycerol backbone. DGAT1 catalyses the attachment of a fatty acid onto the third position of the glycerol backbone. C4:0 is generally attached to the third position of the triglyceride, and is thus usually catalyzed by DGAT1 (numbers preceding the colon represent the number of carbon atoms in the fatty acid chain, whereas the number following the colon represents the number of double bonds in the chain). If C4:0 is not attached to the third position by DGAT1, levels of C4:0 in cellular pools may increase and result in an increase of C4:0 being placed on position 1 or 2 of the triglyceride.

Human milk triglycerides predominately comprise C16:0 in position 2 of the glycerol backbone, which provides for better solubility on lipolysis by 1,3 lipase, as the 2-monoglyceride containing C16:0 is more soluble than the C16:0 free fatty acid released from the 1 and 3 positions. In bovine milk for example, C16:0 is generally found on positions 1/3 and 2 of the glycerol backbone in about a 1:1 ratio. Milk powder produced from bovines with a mutation of the invention may be found to be more soluble on lipolysis than milk powder produced from bovines carrying only wild-type DGAT1. This is likely to be due to an increase in triglycerides with C16:0 in position 2 of the glycerol backbone. An increase in triglycerides with C12:0 and C14:0 in position 2 is also expected. Bovine milk with triglycerides having C16:0 and C14:0 in position 2 of the glycerol backbone is more like human milk, and thus would be beneficial to consumers, including human infants.

Milk produced by an animal, including a bovine, with a mutation of the invention maintains a typical dairy protein composition. Thus, there are no additional procedures required to process the protein streams.

Since the total milk fat content produced by bovine carrying a mutation of the invention is decreased, and the protein content remains normal, there is a shift in the ratio of milk fat:protein, i.e. there is a substantial decrease in the milk fat:protein ratio in bovine carrying a mutation of the invention, including the exemplified A8078C mutation.

The milk fat derived from an animal with a mutation of the invention may have an increased concentration of fat-soluble compounds, such as vitamins and flavours. This may result in more intense flavours because there is a lower ratio of fat:fat soluble compounds. Interestingly, the generally unwelcome flavour of milk derived from pasture fed bovines is diluted in milk derived from a bovine with the A8078C mutation encompassed by the present invention.

Milk fat derived from an animal with a mutation of the invention also has a decreased fat hardness. This is indicated by a reduced solid fat content of the extracted milk fat at 10° C.

The present invention also relates to products produced from the milk of an animal carrying a mutation of the invention. Such products include, but are not limited to, dairy products such as ice creams, yoghurts and cheeses, dairy based drinks such as milk drinks including milk shakes, and yoghurt drinks, milk powders, dairy based sports supplements, as well as food additives such as protein sprinkles and dietary supplement products, including daily supplement tablets.

A softer dairy fat product can be produced from milk obtained from an animal with a mutation of the invention compared with an animal carrying only wild-type DGAT1. The consistency and texture of the dairy fat is closer to that of plant oils than the dairy fat derived from an animal carrying only wild-type DGAT1.

Dairy fat obtained from animals carrying a mutation of the invention has a lower melting temperature than the dairy fat derived from an animal carrying only wild-type DGAT1. Dairy fat obtained from animals carrying a mutation of the invention may be mixed with fats of a higher melting temperature to depress the melting temperature from the higher melting temperature, i.e. to an intermediate melting temperature. The texture of the fats would be improved (softer) from that of the fats on their own.

The softer dairy fat products obtained from animals carrying a mutation of the invention retain and/or enhance the “dairy” flavour and texture. Softer dairy fat products made in a technical or synthetic way tend to lose the “dairy” flavour and texture of such products made directly from milk fats. Softer dairy fat products include, for example, cottage cheese, cream cheese, soft cheese, and whipping creams. For example it has been shown that butter manufactured from milk obtained from animals carrying the 8078C mutation is spreadable at a lower temperature than butter derived from a bovine carrying only wild-type DGAT1.

Products that may be made from milk produced from an animal with a mutation of the invention will generally have a lower fat content than products made from milk from an animal carrying only wild-type DGAT1, because of the reduction in the percentage of fat in milk produced by the animal carrying the mutation. Thus, lower fat milk may be produced without requiring steps in the manufacturing process to remove milk fats. Similarly, other lower fat dairy and/or lower saturated fat products may also be produced. Human consumption of lower fat and/or lower saturated fat foods may avoid health conditions associated with high fat diets, such as cardiovascular diseases including coronary (or ischaemic) heart disease, cerebrovascular disease, hypertension, heart failure and rheumatic heart disease.

The fat content of milk produced from an animal with a mutation of the invention may be further lowered by feeding the animal a diet comprising oil-containing feed, such as seeds or pasture plants. This would also result in an increased level of unsaturated fatty acids. Alternatively, or additionally, the animal may be administered conjugated linoleic acid (CLA) to further depress milk fat synthesis and saturation.

In one embodiment of the present invention, products (as described above) may be made from milk obtained from an animal that carries a mutation of the present invention, and which carries a K232A amino acid substitution in the same allele of DGAT1. For example, it has been shown that one chromosome of the bovine carrying the A8078C mutation of the invention, and the low milk fat daughters of this bovine, carries an allele encoding a DGAT1 polypeptide lacking the 21 amino acids encoded by exon 16 and containing the 232A substitution.

In one embodiment, a bovine carrying the A8078C nucleotide mutation produces milk, or is capable of producing progeny that produce milk, with less than about 3% total milk fat in their milk, at least about 27% unsaturated fatty acids in the total milk fatty acid content of their milk, less than about 57% saturated fatty acids in the total milk fatty acid content of their milk, and/or at least about 1.2% of omega-3 fatty acids in the total milk fatty acid content of their milk. Still further, a bovine carrying the A8078C nucleotide mutation produces milk, or is capable of producing progeny that produce milk, at a volume of about 6000 litres per season under management regimes similar to New Zealand's pasture based farming, namely dairy cattle grazing rye grass/white clover pasture. It is possible that a higher volume could be achieved by a bovine carrying the A8078C nucleotide mutation farmed under a different farming system.

As used herein, an “advantageous tissue profile” refers to tissue obtained from the animal having at least one or more qualities selected from the group consisting of a reduction in total fat as a percentage of total mass, an increase in the percentage of unsaturated fatty acids in the total fatty acid content, a decrease in the percentage of saturated fatty acids in the total fatty acid content, an increase in protein yield, an increase in the percentage of omega-3 fatty acids in the total fatty acid content, a decrease in fat hardness as indicated by a reduced solid fat content of extracted fat at 10° C., a decrease in the ratio of fat:protein, and an increase in the volume of meat produced due to a general increased growth rate of the animal.

The invention also relates to tissues and tissue products derived from an animal carrying a mutation of the invention, including, but not limited to, meat, organs, pelts, fluids, for example blood and serum, and the like. These tissues typically have a decreased fat content and/or decreased degree of fat saturation.

As used herein, the term “increased growth rate” refers to the rate of increase in weight or size dyer time, the time required to reach a defined target weight or size, and/or the time to reach sexual maturity.

The present invention also encompasses variants of the nucleic acid molecules and polypeptides of the present invention. The term “variant” as used herein refers to a nucleic acid molecule or polypeptide having nucleotide or amino acid sequences, respectively, that are different from the specifically identified sequences, but which preserve the functional equivalence of those sequences. For example, a variant nucleic acid molecule may encompass a nucleic acid molecule that differs from the sequences of the invention but that, as a consequence of the degeneracy of the genetic code, encodes a polypeptide having similar activity to a mutant polypeptide encoded by a nucleic acid molecule of the present invention. A nucleotide sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Nucleotide sequence alterations resulting in conservative substitutions of one or several amino acids in the encoded polypeptide sequence are also included in the invention. A skilled artisan will be aware of methods for making phenotypically silent amino acid substitutions (see, e.g. Bowie et al., 1990, Science 247: 1306).

It would be understood that the identification of a mutation of the invention enables methods of assessing the genetic merit of an animal, for example a bovine, with respect to an advantageous milk profile (more particularly milk fat composition), an advantageous tissue profile and/or an increased growth rate.

As used herein, the term “genetic merit” refers to the sum of all positive and negative genetic effects on a given phenotypic trait. Estimated genetic merit is typically expressed as the estimated breeding value of a cow or a bull for a given phenotypic trait.

The identification of a mutation of the invention also enables methods for selecting an animal, such as a bovine, that produces an advantageous milk profile, an advantageous tissue profile, an advantageous colostrum profile, and/or an increased growth rate. This in turn, for example, may allow the selection of milk with desired milk fat composition to be directed to production of suitable milk products. For example, milk produced by bovine selected for producing milk with lower percentage of total milk fat and a higher percentage of unsaturated fatty acids may be directed to production of low-fat milk products, such as low-fat yoghurt. Milk produced by bovine selected for producing milk with a lower percentage of saturated fatty acids and/or a higher percentage of unsaturated fatty acids may also be directed to production of products which are well-known to be traditionally high in fat, such as ice creams, providing a healthier alternative to products with a higher percentage of saturated fats.

The identification of a mutation of the invention also enables methods for determining a DGAT1 genotype of an animal with respect to a mutation of the invention.

The aforementioned methods include determining if the bovine comprises a DGAT1 nucleic acid molecule or polypeptide, as described above, having a mutation of the invention. Alternatively, the DGAT1 exon 16 allelic profile of the animal may be determined. Means for performing such methods are known in the art. The following paragraphs will provide examples, with reference in-part to the A8078C DGAT1 nucleotide mutation which has been identified by the inventors in bovine.

### 1 Identification of Nucleic Acid Molecules and Polypeptides Having a Mutation of the Invention

There are numerous standard methods known in the art for determining if an animal comprises a DGAT1 nucleic acid molecule or polypeptide having a mutation of the invention. For example, such methods may include the step of sequencing a nucleic acid molecule (e.g. DNA) sample taken from the animal. Thus in one embodiment of the invention, the step of determining whether or not an animal comprises a nucleic acid molecule having a mutation of the invention includes the step of sequencing a nucleic acid molecule obtained from the animal. Methods for nucleotide sequencing are well known to those skilled in the art.

An example of another standard method for determining whether a particular nucleic acid molecule is present in an animal is the Polymerase Chain Reaction (PCR) (Mullis et al., Eds. 1994. The Polymerase Chain Reaction, Birkhauser). Oligonucleotide primers which flank and/or incorporate a mutation of the invention may be used to amplify a nucleic acid molecule in a sample obtained from the animal under test. The nucleic acid molecule may be selected from genomic DNA, RNA (e.g. mRNA or hnRNA), or cDNA produced from mRNA (see Sambrook J et al., 2001, supra for general methods for cDNA production).

The oligonucleotide primers will have sufficient complementarity to the DGAT1 nucleotide sequence, and be of sufficient length to selectively hybridise to a DGAT1 nucleic acid molecule intended to be amplified, thereby priming DNA synthesis under in vitro conditions commonly used in PCR. In one example, one of the primers used in the PCR amplification step may recognise and bind to only a mutated sequence of DGAT1. Presence of a PCR product will therefore indicate that a nucleic acid molecule present in the sample, and therefore the animal, has the mutation. In another example, the PCR amplification step may use primers which flank the mutation, for example one primer may bind to sequences in exon 15, and the other primer may bind to sequences in exon 17 of DGAT1. Amplification from cDNA (produced from mRNA) will then identify mutations which give rise to alternate splicing of exon 16, such as the A8078C nucleotide mutation encompassed by the present invention. A PCR product smaller in size than that expected will indicate deletion of DGAT1 coding sequences, whereas a PCR product larger in size than expected indicates an insertion mutation.

Primers suitable for use in PCR based methods of the invention comprise at least about 10 contiguous nucleotides of, or complementary to, the nucleotide sequences set forth in one of SEQ ID NOs:1, 2, 43 and 44, or naturally occurring flanking sequences thereof. Examples of PCR primers which may be used for the aforementioned methods are presented herein as SEQ ID NO: 5, SEQ ID NO: 6 and SEQ ID NO:7.

Other PCR-based methods include reverse-transcriptase PCR-based applications which can be used to detect mutations of the invention which disrupt transcription, and therefore the expression, of DGAT1. For example, quantitative RT-PCR may be used in which nucleic acid molecules in, the form of mRNA are obtained from the animal being tested, and are reverse transcribed. Real-time PCR is then performed using oligonucleotide primers specific for DGAT1 and other (control) genes expressed in the same sample, and the level of DGAT1-specific transcripts is normalized relative to that of the control gene(s) in, order to establish a value of expression of DGAT1. The values obtained are compared to expression values obtained from animals expressing wild-type. DGAT1. A reduction in expression of DGAT1 will indicate the presence of a mutation of the invention which disrupts transcription of DGAT1. Other quantitative amplification methods well known in the art may also be employed, and include for example microarray analysis.

Other methods for determining whether a particular nucleic acid molecule is present in an animal may include the step of restriction enzyme digestion of a nucleic acid molecule sample taken from the animal. For example, a mutation of the invention may create or destroy an endonuclease restriction site. Therefore, separation and visualisation of digested restriction fragments by methods well known in the art, may form a diagnostic test for the presence or absence of a particular nucleotide sequence. The nucleotide sequence digested may be a PCR product amplified as described above.

For example, the A8078C mutation destroys a NlaIII restriction site in the wild-type sequence, and at the same time creates a new HaeIII site in the mutant sequence. Accordingly, digestion of DGAT1 DNA which contains the A8078C mutation with NlaIII or HaeIII will result in different sized restriction fragments when compared to wild-type DGAT1 DNA. Such altered restriction fragment sizes may be detected by standard methodologies known in the art such as agarose gel electrophoresis and/or Southern hybridisation.

Still other methods for determining whether a particular nucleic acid molecule is present in an animal may include the step of hybridisation of a probe to a nucleic acid molecule sample taken from the animal. Such probes should comprise a nucleic acid molecule of sufficient length and sufficient complimentarity to the DGAT1 nucleotide sequence, to selectively bind under high or low stringency conditions with DGAT1 nucleic acid molecules contained in the nucleic acid molecule sample to facilitate detection of the presence or absence of a mutation of the invention. With respect to probes greater than about 100 nucleotides in length, typical stringent hybridization conditions are no more than 25 to 30° C. (for example, 10° C.) below the melting temperature (Tm) of the native duplex (see generally, Sambrook J et al., 2001, supra; Ausubel et al., 1987, Current Protocols in Molecular Biology, John Wiley & Sons, Inc). The Tm for nucleic acid molecules greater than about 100 nucleotides in length can be calculated by the following formula: Tm=81.5+0.41% (G+C-log (Na+)). Typical stringent conditions for polynucleotides of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C. With respect to probes having a length less than about 100 nucleotides, exemplary stringent hybridization conditions are generally 5 to 10° C. below the Tm. On average, the Tm of a nucleic acid molecule of length less than 100 nucleotides is reduced by approximately (500/nucleic acid molecule length)° C. With respect to the DNA mimics known as peptide nucleic acids (PNAs) (Nielsen et al., 1991, Science 254(5037):1497-500) Tm values are higher than those for DNA-DNA or DNA-RNA hybrids, and can be calculated using the formula described in Giesen et al., 1998 Nucleic Acids Res. 26(21):5004-5006. Exemplary stringent hybridization conditions for a DNA-PNA hybrid having a length less than 100 bases are 5 to 10° C. below the Tm.

The nucleic acid molecule sample taken from the animal may be genomic DNA or RNA (including mRNA or hnRNA). The analysis may also be conducted on cDNA produced from mRNA.

The aforementioned probes would typically comprise at least 10 contiguous nucleotides of, or complementary to, the nucleotide sequence set forth in one of SEQ ID NOs:1, 2, 43 and 44, or naturally occurring flanking sequences thereof. Accordingly, the probe may comprise a nucleotide sequence which includes a mutation of the invention.

Such probes may additionally comprise means for detecting the presence of the probe when bound to nucleic acid molecule sample. Methods for labelling probes such as radiolabelling are well known in the art (see for example, Sambrook J et al., 2001, supra).

One example of a probe-based detection assay is Northern analysis using probes able to hybridise to the target DGAT1 mRNA. As would be evident to a person skilled in the art, Northern analysis is a quantitative method that requires normalisation of sample concentrations with respect to each other. This is generally achieved by normalising the samples with respect to an internal control, such as the amount of rRNA present in each sample.

The aforementioned methods of the present invention are reliant on genetic information such as that derived from methods, including those described above, suitable for the detection and identification of polymorphisms, particularly single nucleotide polymorphisms (SNPs), associated with the trait for which an assessment is desired. For the sake of convenience the following discussion refers particularly to SNPs, however a person skilled in the art will appreciate that the methods discussed are amenable to the detection and identification of other genetic polymorphisms, such as triplet repeats or microsatellites.

A SNP is a single base change or point mutation resulting in genetic variation between individuals. SNPs are believed to occur in mammalian genomes approximately once every 100 to 300 bases, and can occur in coding or non-coding regions. Due to the redundancy of the genetic code, a SNP in the coding region may or may not change the amino acid sequence of a protein product. A SNP in a non-coding region can, for example, alter gene expression by, for example, modifying control regions such as promoters, transcription factor binding sites, processing sites, ribosomal binding sites, mRNA stability, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, drug responsiveness including, for example, susceptibility to adverse drug reactions, and as described herein association with desirable phenotypic traits. Knowledge of the association of a particular SNP with a phenotypic trait, coupled with the knowledge of whether a subject has said particular SNP, can enable the targeting of diagnostic, preventative and therapeutic applications to allow better disease management, to enhance understanding of disease states, to develop selective breeding regimes, and to identify subjects of desirable genetic merit.

Indeed, a number of databases have been constructed of known SNPs, and for some such SNPs, the biological effect associated with a SNP. Understandably, there has been a focus on human genetics. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait. Similar databases are available for a number of species of commercial and scientific interest.

There has been and continues to be a great deal of effort to develop methods that reliably and rapidly identify new SNPs associated with a phenotypic trait. This is no trivial task, at least in part because of the complexity of mammalian genomic DNA (e.g., the haploid human genome of 3×10⁹ base pairs, while current estimates of the size of the haploid bovine genome are in the range of 2.7-2.9×10⁹ base pairs), and the associated sensitivity and discriminatory requirements.

Genotyping approaches to detect SNPs are well-known in the art, and have been described generally above. Such methods include DNA sequencing, methods that require allele specific hybridization of primers or probes, allele specific incorporation of nucleotides to primers bound close to or adjacent to the polymorphisms (often referred to as “single base extension”, or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A, C, G, or T), and a DNA polymerase. Only one of the four nucleotides (homozygous case) or two of the four nucleotides (heterozygous case) is incorporated. The base that is incorporated is complementary to the nucleotide at the SNP position.

A number of methods currently used for SNP detection involve site-specific and/or allele-specific hybridisation. These methods are largely reliant on the discriminatory binding of oligonucleotides to target sequences containing the SNP of interest. The techniques of Affymetrix (Santa Clara, Calif.) and Nanogen Inc. (San Diego, Calif.) are particularly well-known, and utilize the fact that DNA duplexes containing single base mismatches are much less stable than duplexes that are perfectly base-paired. The presence of a matched duplex is detected by fluorescence.

The majority of methods to detect or identify SNPs by site-specific hybridisation require target amplification by methods such as PCR to increase sensitivity and specificity (see, for example U.S. Pat. No. 5,679,524, PCT publication WO 98/59066, PCT publication WO 95/12607). US Application 20050059030 (incorporated herein in its entirety) describes a method for detecting a single nucleotide polymorphism in total human DNA without prior amplification or complexity reduction to selectively enrich for the target sequence, and without the aid of any enzymatic reaction. The method utilises a single-step hybridization involving two hybridization events: hybridization of a first portion of the target sequence to a capture probe, and hybridization of a second portion of said target sequence to a detection probe. Both hybridization events happen in the same reaction, and the order in which hybridisation occurs is not critical.

US Application 20050042608 (incorporated herein in its entirety) describes a modification of the method of electrochemical detection of nucleic acid hybridization of Thorp et al. (U.S. Pat. No. 5,871,918). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labelled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP. This technique varies in how rapidly it can be performed, from a few samples per day to a high throughput of 40,000 SNPs per day, using mass code tags. A preferred example is the use of mass spectrometric determination of a nucleic acid molecule which comprises a mutation of the invention; including for example the A8078C mutation. Such mass spectrometric methods are known to those skilled in the art, and the genotyping methods of the invention are amenable to adaptation for the mass spectrometric detection of the polymorphisms of the invention.

The presence of particular SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′ end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174.

U.S. Pat. No. 6,821,733 (incorporated herein in its entirety) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Furthermore, a large number of methods reliant on the conformational variability of nucleic acids have been developed to detect SNPs. For example, Single Strand Conformational Polymorphism (SSCP) (Orita et al., 1989, PNAS 86:2766-2770) is a method reliant on the ability of single-stranded nucleic acids to form secondary structure in solution under certain conditions. The secondary structure depends on the base composition and can be altered by a single nucleotide substitution, causing differences in electrophoretic mobility under non-denaturing conditions. The various polymorphs are typically detected by autoradiography when radioactively labelled, by silver staining of bands, by hybridisation with detectably labelled probe fragments or the use of fluorescent PCR primers which are subsequently detected, for example by an automated DNA sequencer.

Modifications of SSCP are well known in, the art, and include the use of differing gel running conditions, such as for example differing temperature, or the addition of additives, and different gel matrices. Other variations on SSCP are well known to the skilled artisan, including, RNA-SSCP, restriction endonuclease fingerprinting-SSCP, dideoxy fingerprinting (a hybrid between dideoxy sequencing and SSCP), bi-directional dideoxy fingerprinting (in which the dideoxy termination reaction is performed simultaneously with two opposing primers), and Fluorescent PCR-SSCP (in which PCR products are internally labelled with multiple fluorescent dyes, may be digested with restriction enzymes, followed by SSCP, and analysed on an automated DNA sequencer able to detect the fluorescent dyes).

Other methods which utilise the varying mobility of different nucleic acid structures include Denaturing Gradient Gel Electrophoresis (DGGE), Temperature Gradient Gel Electrophoresis (TGGE), and Heteroduplex Analysis (HET). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilised to detect SNPs, using HPLC methods well-known in the art as an alternative to the separation methods described above (such as gel electophoresis) to detect, for example, homoduplexes and heteroduplexes which elute from the HPLC column at different rates, thereby enabling detection of mismatch nucleotides and thus SNPs.

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly used in Maxam-Gilbert sequencing chemistry, are all well known in the art.

Further examples include the Protein Translation Test (PTT), used to resolve stop codons generated by variations which lead to a premature termination of translation and to protein products of reduced size, and the use of mismatch binding proteins. Variations are detected by binding of, for example, the MutS protein, a component of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

The aforementioned methods of detecting and identifying SNPs are amenable to the identification of a nucleic acid molecule having a mutation of the invention, and accordingly may be used in the methods of the invention.

Protein- and proteomics-based approaches are also suitable for detection and analysis of polypeptides containing a mutation of the invention. Mutations which result in, or are associated with, variation in expressed polypeptides can be detected directly by analysing said polypeptides. This typically requires separation of the various proteins within a sample obtained from an animal being tested, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomIQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilise mass spectrometry, including ion trap mass spectrometry, liquid chromatography (LC) and LC/MS mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining mutations that result in or are associated with variation in post-translational modifications of proteins.

Associated technologies are also well known, and include, for example, protein processing devices such as the “Chemical Inkjet Printer” comprising piezoelectric printing technology that allows in situ enzymatic or chemical digestion of protein samples electroblotted from 2-D PAGE gels to membranes by jetting the enzyme or chemical directly onto the selected protein spots. After in situ digestion and incubation of the proteins, the membrane can be placed directly into the mass spectrometer for peptide analysis.

Other suitable polypeptide-based analyses include, but are not limited to, Native polyacrylamide gel electrophoresis (PAGE), isoelectric focussing, 2D PAGE, Western blotting with specific antibodies, immunoprecipitation, and peptide fingerprinting.

As indicated above, the identification of a mutation of the invention enables methods for determining the genetic merit of an animal or for selecting an animal with advantageous, milk, tissue and/or growth rate properties. Such methods may rely on determination of the DGAT1 exon 16 allelic profile and/or genotype of the animal.

The allelic profile of exon 16 of DGAT1 may be determined with reference to the nucleotide composition of one (haplotype) or both (genotype) alleles of the DGAT1 gene. For example, with reference to the A8078C mutation in DGAT1, the wild-type allele comprises an adenine at position 8078, and is therefore referred to herein as the “A allele”. The A allele preferably comprises the nucleotide sequence set forth in SEQ ID NOs:1 or 43. The mutated allele of DGAT1 comprises a cytosine at position 8078, and is therefore referred to herein as the “C allele”. The C allele preferably comprises the nucleotide sequence of SEQ ID NOs: 2 or 44. Therefore, the particular nucleotide at position 8078 of DGAT1 will determine the allelic profile of DGAT1, and in particular, the allelic profile of exon 16 of DGAT1.

Identification of the presence of a particular nucleotide composition of exon 16 of DGAT1 (i.e. identification of the presence of a mutation of the invention) in order to determine the allelic profile of the exon in an animal, can be achieved in a number of ways, as described above. For example, the presence of a particular mutation of the invention, including the A8078C mutation, can be determined by techniques such as single strand conformation polymorphism analysis (SSCP) or the like. The presence of a mutation of the invention can also be determined by directly sequencing nucleic acid molecules obtained from the animal. Alternatively, restriction enzyme digestion may be employed. Furthermore, PCR and reverse transcriptase PCR may be used to amplify DNA or mRNA, respectively, obtained from the animal in order to establish the allelic profile of exon 16. For example, one of the primers used in the PCR amplification step may recognise and bind to only a wild-type or bind to only a mutated sequence of exon 16 of DGAT1. Presence or absence of a PCR product may therefore elucidate the allelic profile. PCR amplification of mRNA (or cDNA obtained from mRNA) may identify deletion or insertion mutations if PCR primers binding either side of exon 16 are used in the amplification reaction. For example, one primer may bind to sequences in exon 15 of DGAT1, and the other primer may bind to sequences in exon 17 of DGAT1. PCR amplification will then identify mutations which give rise to alternate splicing of exon 16, such as the A8078C nucleotide mutation encompassed by the present invention. A PCR product smaller in size than that expected will indicate deletion of DGAT1 coding sequence, whereas a PCR product larger in size than expected indicates an insertion mutation: Other methods of identifying the presence of a mutation of the invention, and therefore determining the exon 16 allelic profile of DGAT1 would be known in the art, and are described above. These include mass spectrometric analysis of nucleic acid molecules (e.g. DNA or mRNA) obtained from the animal, or Southern analysis of DNA or mRNA using probes which recognise and bind to a particular allele in exon 16.

Determination of the allelic profile of the exon in an animal can also be achieved by analysis of a polypeptide sample obtained from the animal, such methods being described above. For example, the A allele of DGAT1 is part of a codon which encodes a methionine amino acid in exon 16. The presence of the methionine may be determined by directly sequencing the polypeptide obtained from the animal.

Genetic loci which are linked to, or are in linkage disequilibrium with, a mutation of the invention may also be used to (indirectly) determine the presence of the mutation, thereby also determining the exon 16 allelic profile of DGAT1. For example, several markers linked to, or in linkage disequilibrium with, the nucleic acid molecule set forth in SEQ ID NO. 2 have been identified by the inventors, for example, ARS-BFGL-NGS-4939, Hapmap52798-ss46526455, Hapmap29758-BTC-003619, BFGL-NGS-18858, Hapmap24717-BTC-002824, and Hapmap24718-BTC-002945. Linkage is a phenomenon in genetics whereby two or more mutations or polymorphisms are located on the same chromosome and are close enough to be generally co-inherited. Genetic association, or linkage disequilibrium, is a phenomenon in genetics whereby two or more mutations or polymorphisms are in such close genetic proximity that they are co-inherited at a high frequency. This means that in genotyping, detection of one polymorphism as present implies the presence of the other (Reich D E et al., 2001, Nature 411:199-204.)

Furthermore, animals may be screened for both a mutation of the present invention and other polymorphisms in the same or different genes, including the polymorphism in DGAT1 encoding the K232A amino acid substitution, and the F279Y amino acid mutation in the GHR gene. The combination of a mutation of the invention together with one or more other polymorphisms may provide a synergistic effect. A mutation of the invention may be present on the same or different chromosome as one or more other polymorphisms. The animal may be homozygous or heterozygous for a mutation of the invention and homozygous or heterozygous for a polymorphism in one or more other genes.

The mutated DGAT1 allele identified by the inventors has the nucleotide sequence set forth in SEQ ID NOs:2 or 44. The sequence set forth in SEQ ID. NO:2 encodes an alanine residue at position 232 of the DGAT1 polypeptide, whereas the sequence set forth in SEQ ID NO:44 encodes a lysine, residue at position 232 of the DGAT1 polypeptide. As indicated above, the K232A polymorphism arises due to a di-nucleotide substitution in DGAT1, wherein the AA nucleotides at positions 694 and 695 of the coding region of DGAT1 (i.e. positions 6829 and 6830 of GenBank Accession AY065621/GI:18642597) are substituted for GC. It is expected that the combination of the 232A polymorphism and the deletion of the 21 amino acids encoded by exon 16 of DGAT1 will have a synergistic effect with respect to an advantageous milk profile. Therefore, a synergistic effect may be achieved in an animal having a genotype including one mutated allele, i.e. one copy of the DGAT1 allele with the nucleotide sequence set forth in SEQ ID NO: 2 or 44, and one allele coding for a wild-type DGAT1 polypeptide. In this example, an animal with at least one mutated DGAT1 allele could have one of the following DGAT1 genotypes: 6829G 6830C/6829G 6830C, 8078A/8078C; 6829G 6830C/6829A 6830A, 8078C/8078A; 6829A 6830A/6829A 6830A, 8078A/8078C; 6829G 6830C/6829G 6830C, 8078C/8078C; 6829A 6830A/6829A 6830A, 8078C/8078C. The alleles constituting these genotypes have the nucleotide and amino acid sequences as set out in the Table 1 below.

TABLE 1

| | Nucleotide | Amino Acid | Wild-type |
|---|---|---|---|
| Allele | Sequence | Sequence | or Mutant |
| 6829G 6830C 8078A | SEQ ID NO: 1 | SEQ ID NO: 3 | Wild-type |
| 6829A 6830A 8078A | SEQ ID NO: 43 | SEQ ID NO: 45 | Wild-type |
| 6829G 6830C 8078C | SEQ ID NO: 2 | SEQ ID NO: 4 | Mutant |
| | | (M435L) or | |
| | | SEQ ID NO: 47 | |
| | | (Δ418-438) | |
| 6829A 6830A 8078C | SEQ ID NO: 44 | SEQ ID NO: 46 | Mutant |
| | | (M435L) or | |
| | | SEQ ID NO: 48 | |
| | | (Δ418-438) | |

As indicated above, the present invention enables methods for assessing the genetic merit, or determining the genotype, of an animal (for example a bovine), with respect to an advantageous milk, tissue or growth rate profile. In one embodiment, these methods include determining if the animal comprises a polypeptide, or comprises a nucleic acid molecule encoding a polypeptide, having: (i) biological activity of wild-type DGAT1 (i.e. the animal comprises polypeptide (A) or nucleic acid molecule (A)); or (ii) having a DGAT1 amino acid sequence with a mutation in one or more of the amino acids encoded by exon 16 of DGAT1 (i.e. the animal comprises polypeptide (B) or nucleic acid molecule (B)); or (iii) a combination of (i) and (ii). In this instance, and with reference to Table 1 above, nucleic acid molecule (A) may have the nucleotide sequence set forth in SEQ ID NOs: 1 or 43, whereas nucleic acid molecule (B) may have the nucleotide sequence set forth in SEQ ID NOs: 2 or 44. Furthermore, polypeptide (A) may have the amino acid sequence set forth in SEQ ID NOs: 3 or 45, and polypeptide (B) may have the amino acid sequence set forth in one of SEQ ID NOs: 4, 46, 47 and 48. Methods for determining which DGAT1 nucleic acids and polypeptides the animal comprises have been described above.

### 2 Diagnostic Kits

The present invention further provides diagnostic kits useful for detecting a nucleic acid molecule of the present invention, such as for determining the exon16 DGAT1 allelic profile and/or genotype of an animal under test, and for use in other methods of the present invention, as described above.

Accordingly, in one embodiment the invention provides a diagnostic kit which can be used to determine the DGAT1 genotype of an animal, including a bovine. A diagnostic kit may include a set of primers which amplify DGAT1 from a sample of nucleic acid molecules obtained from the animal. The primers will typically include nucleotide sequences which amplify a region of the DGAT1 gene containing a mutation of the invention. For example, the actual genotyping may be carried out using a primer that targets a specific mutation of the invention, and that can function as an allele-specific oligonucleotide in conventional hybridisation, Taqman assays, OLE assays, etc. Alternatively, primers can be designed to permit genotyping by microsequencing.

Accordingly, one kit of primers can include first, second and third primers, (a), (b) and (c), respectively. Primer (a) is complementary to, and therefore binds to, a region containing a DGAT1 mutation of the invention. Primer (b) is complementary to, and therefore binds to, a region upstream or downstream of the region to, be amplified, by, a primer (a) so that genetic material containing the mutation is, amplified, by PCR, for example, in the presence of the two primers. Primer (c) is, complementary to, and therefore binds to, the region corresponding to that which primer (a) binds, but primer (c) lacks the mutation, i.e. it includes the wild-type nucleotide. Thus, genetic material containing the non-mutated region will be amplified in the presence of primers (b) and (c). Genetic, material homozygous for the wild-type gene will thus provide amplified products in the presence of primers (b) and (c). Genetic material homozygous for the mutated gene will thus provide amplified products in the presence of primers (a) and (b). Heterozygous genetic material will provide amplified products in both cases.

In one embodiment, the diagnostic kit is useful in detecting DNA comprising a DGAT1 gene or encoding a DGAT1 polypeptide containing a mutation of the invention. The kit may include first and second primers for amplifying the DNA, the primers being complementary to nucleotide sequences of the DNA upstream and downstream, respectively, of the mutation which results in an advantageous milk, tissue and/or growth rate profile. In one embodiment, at least one of the nucleotide sequences is selected to be complementary, and therefore hybridises to, a non-coding region of the DGAT1 gene. For example, in one embodiment, the kit may include oligonucleotide primers with the sequences set forth in SEQ ID NOs: 5 and 6. The kit can also include a third primer which is complementary, and therefore binds to, the mutation.

Preferably the kit includes instructions for use, for example in accordance with a method of the invention.

In one embodiment, the diagnostic kit comprises a nucleotide probe which is complementary to, and therefore binds to, the nucleotide sequence set forth in one of SEQ ID NOs:1, 2, 43 and 44. The probe may for example, hybridise with DNA or mRNA obtained from the animal being tested. The kit may include means for detecting the nucleotide probe bound to mRNA in the sample, such means being known in the art. In a particular aspect, the kit of this aspect of the invention includes a probe having a nucleic acid molecule sufficiently complementary with the nucleotide sequence set forth in one of SEQ ID NOs:1, 2, 43 and 44, so as to bind thereto under stringent conditions. “Stringent” hybridisation conditions takes on its common meaning to a person skilled in the art. Appropriate stringency conditions which promote nucleic acid hybridisation depend on the length of the probe, and are for example, 6× sodium chloride/sodium citrate (SSC) at about 45° C. Appropriate wash stringency depends on the degree of homology and the length of the probe. If homology between the probe and target sequence is 100%, a high temperature (65° C. to 75° C.) may be used. However, if the probe is very short (<100 bp), lower temperatures must be used even with 100% homology. In general, one starts washing at low temperatures (37° C. to 40° C.), and raises the temperature by 3-5° C. intervals until background is low enough to not interfere with autoradiography. The diagnostic kit can also contain an instruction manual for use of the kit.

In another embodiment, the diagnostic kit comprises an antibody, as described below, or an antibody composition useful for detection of the presence or absence of wild type DGAT1 and/or the presence or absence of a polypeptide containing a mutation of the invention.

### 3 Antibodies

The present invention also provides antibodies, and compositions thereof, which detect a polypeptide of the present invention. Such antibodies may include, but are not limited to, polyclonal, monoclonal, chimeric and single chain antibodies. For the production of antibodies, various hosts including rabbits, rats, goats, mice, humans, and others may be immunized by injection with a polypeptide of the invention, or with any fragment or oligopeptide thereof, which has immunogenic properties. Various adjuvants may be used to increase immunological response and include, but are not limited to, Freund's, mineral gels such as aluminum hydroxide, and surface-active substances such as lysolecithin. Adjuvants used in humans include BCG (bacilli Calmette-Guerin) and Corynebacterium parvum. It is preferred that the polypeptide, or fragment or oligopeptide thereof used to induce antibody production have an amino acid sequence consisting of at least 5 amino acids, and, more preferably, of at least 10 amino acids. It is also preferable that the polypeptide, or fragment or oligopeptide thereof are identical to a portion of the amino acid sequence of the natural protein and contain the entire amino acid sequence of a small, naturally occurring molecule. Short stretches of DGAT1 amino acids may be fused with those of another protein, such as KLH, and antibodies to the chimeric molecule may be produced.

Monoclonal antibodies to a polypeptide of the invention may be prepared using any technique which provides for the production of antibody molecules by continuous cell lines in culture. These include, but are not limited to, the hybridoma technique, the human B-cell hybridoma technique, and the EBV-hybridoma technique. (For example, see Kohler G and Milstein C, 1975, Nature 256:495-497; Kozbor D et al., 1985, J. Immunol. Methods 81:31-42; Cote R J et al., 1983, Proc. Natl. Acad. Sci, USA 80:2026-2030; Cole S P et al., 1984, Mol. Cell Biol. 62:109-120). Antibodies may also be produced by inducing in vivo production in the lymphocyte population or by screening immunoglobulin libraries or panels of highly specific binding reagents as disclosed in the literature. (For example, see Orlandi R et al., 1989, Proc. Natl. Acad. Sci. USA 86:3833-3837; Winter G et al., 1991, Nature 349:293-299).

Antibody fragments which contain specific binding sites for a polypeptide of the invention may also be generated. For example, such fragments include, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (For example, see Huse W D et al., 1989, Science 246:1275-1281).

Various immunoassays may, be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between a polypeptide of the invention and its specific antibody. A two-site, monoclonal-based immunoassay utilizing monoclonal antibodies reactive to two non-interfering DGAT1 epitopes is preferred, but a competitive binding assay may also be employed. Diagnostic assays for DGAT1 polypeptides of the invention include methods that utilize the antibody and a label to detect the polypeptide in body fluids or in extracts of cells or tissues. The antibodies may be used with or without modification, and may be labelled by covalent or non-covalent attachment of a reporter molecule.

### 4 Sample Preparation

As will be apparent to persons skilled in the art, samples suitable for use in the methods of the present invention may be obtained from tissues or fluids as convenient; and so that the sample contains the moiety or moieties to be tested. For example, where nucleic acid is to be analysed, tissues or fluids containing nucleic acid will be used.

Conveniently, samples may be taken from milk, tissues, blood, serum, plasma, cerebrospinal fluid, urine, semen, hair or saliva. Tissue samples may be obtained using standard techniques such as cell scrapings or biopsy techniques. For example, the cell or tissue samples may be obtained by using an ear punch to collect ear tissue from the animal. Similarly, blood sampling is routinely performed, for example for pathogen testing, and methods for taking blood samples are well known in the art. Likewise, methods for storing and processing biological samples are well known in the art. For example, tissue samples may be frozen until tested if required. In addition, one of skill in the art would realize that some test samples would be more readily analyzed following a fractionation or purification procedure, for example, separation of whole blood into serum or plasma components.

As described above, the aforementioned methods for detecting a mutation of the invention may be used to select individual bovine, or indeed a herd of bovine. For example, individual bovine containing a mutation of the invention are selected and segregated from those bovine not containing the mutation. The selected bovine are then collected to form the herd.

Additionally, the present invention is directed towards semen, eggs, and nuclei produced by the animals selected by methods of the invention. The semen, eggs and nuclei are useful in further breeding programs.

The present invention is also directed towards milk produced by the animals selected by methods of the present invention, and products produced from such milk.

The present invention also provides for the production of a genetically modified animal, which may include a transgenic animal, comprising a mutation of the invention. Methods for the production of genetically modified animals are known in the art. Such methods include, but are not limited to, generation of a specific mutation of the invention in the DGAT1 gene of the animal, the use of zinc finger nuclease technology (Geurts et al., 2009, Science 325(5939):433), or insertion of a mutant DGAT1 gene (as a genomic or cDNA construct) into the animal by homologous recombination. In this regard, the constructs may include recombination elements (lox p sites) which are recognized by enzymes such as Cre recombinase, and which enhance the recombination process.

By “transgenic animal” is meant an animal that is engineered to contain a mutation of the invention within the cells (some or all of the cells) of the animal. Transgenic animals can be produced by a variety of different methods including transfection, electroporation, microinjection, gene targeting in embryonic stem cells and recombinant viral and retroviral infection (see for example, U.S. Pat. Nos. 4,736,866; 5,602,307; Mullins et al., 1993 Hypertension 22(4):630-633; Brenin et al., 1997 Surg. Oncol. 6(2):99-110; Tuan (ed.), Recombinant Gene Expression Protocols, Methods in Molecular Biology No. 62, Humana Press (1997)). For example, a nucleic acid construct, or “transgenic construct”, which includes a transgene (i.e. a nucleic acid molecule containing a mutation of the invention) that is suitable for use in making a transgenic animal is first made. The construct can include either a cDNA sequence or a genomic sequence that encodes a mutant DGAT1 polypeptide of the invention. In, one embodiment, a bovine genomic DGAT1 coding sequence is used to make the transgenic construct, wherein the construct includes relevant intron and exon sequences found in the wild type bovine DGAT1 gene. To generate a genomic sequence construct encoding a mutant DGAT1 polypeptide of the invention, a bacterial artificial chromosome (BAC), yeast artificial chromosome (YAC), P1 artificial chromosome (PAC) or other chromosomal DNA fragment containing the DGAT1 gene can be used. The desired DGAT1 mutation is introduced into the DGAT1 gene by following protocols that have been documented in the art (see for example, Gong S et al., 2002, Genome Research 12:1992-1998).

The transgenic construct carrying a mutant DGAT1 coding sequence (either a genomic sequence or a cDNA sequence), is then placed in an operable linkage to a promoter that directs expression of DGAT1. The transgenic construct can also include other transcriptional and translational regulatory elements or nucleotide sequences (e.g. cis-acting activators/enhancers or suppressors), and such sequences are operably linked to the polynucleotide which encodes the mutant DGAT1 polypeptide. The promoter and other transcriptional and translational regulatory elements or nucleotide sequences can include those that are native animal sequences which are naturally responsible for expressing DGAT1, or can include sequences of a different origin. For example, a genomic clone carrying a genomic coding region of DGAT1 may include native DGAT1 5′ sequences (including the native promoter region) and 3′ sequences. Alternatively, sequences suitable for use in the practice of the present invention can be sequences of eukaryotic or viral genes, or derivatives thereof, that stimulate or repress transcription of a gene in a specific or non-specific manner, and/or in an inducible (e.g. tetracycline inducible promoter or MMTV steroid-inducible promoter) or non-inducible manner. Examples of promoters that can be employed in the practice of the present invention include, but are not limited to, a prion promoter, a Thy-1 promoter, a PDGF promoter, a tyrosine hydroxylase promoter, a dopamine transporter promoter, a calcium-calmodulin kinase II promoter, an EIA promoter, an MLP promoter, a CMV promoter, an MMLV promoter, an MMTV promoter, a SV40 promoter, a retroviral LTR, a metallothionein promoter, a RSV promoter and the like. The promoter can be a promoter, that directs ubiquitous expression, or expression in a tissue-specific manner, e.g. expression in mammary gland only.

A desirable transgenic nucleic acid construct may then be employed to generate a transgenic animal, for example a transgenic bovine. This can be achieved in a number of ways. In one approach, an embryo at the pronuclear stage is harvested from a female and the transgenic construct is microinjected into the embryo, in which case the transgenic nucleic acid is chromosomally integrated into the genome of the embryo. The modified embryo is implanted in a pseudopregnant female animal which allows the modified embryo to develop to term. The resulting mature animal will contain the genetic modification in both the germ cells (sperm- or egg-producing cells) and somatic cells. In another approach, embryonic stem (ES) cells are isolated from an animal and the transgenic construct is introduced into the cells by electroporation, transfection or microinjection. The transgenic nucleic acid integrates into the genome via non-homologous recombination. The modified ES cells are then implanted into a blastocyst (an early embryo), which is then implanted into the uterus of a female animal. Progeny born from this blastocyst is a chimeric animal, i.e. an animal containing cells derived from the modified ES cells as well as cells derived from the unmodified cells of the blastocyst. By selecting progeny having germ cells developed from the modified cells and interbreeding them, progeny that contain the genetic modification in all of their cells can be obtained. The pronuclear microinjection approach may be especially suitable for introducing large size genomic-type transgenic constructs such as a BAC carrying a genomic polynucleotide which codes for a mutant DGAT1 polypeptide of the invention.

Progeny can be tested for incorporation of the transgene by analysis of tissue samples using transgene-specific probes. Southern blot analysis and PCR are particularly useful in this regard. The expression of a transgene can also be assessed by analysis of levels of mRNA or levels of the mutant DGAT1 polypeptide in tissue samples using appropriate assays, for example, Northern blot analysis and Western blot analysis, among others. Tissue samples for these analyses can include samples obtained from the mammary gland.

As indicated above, transgenic animals carrying a mutation of the invention may also be produced using zinc finger nucleases. This technique is described in Geurts et al., 2009, supra, and does not require the use of embryonic stem cells. Rather, targeted mutations are induced by standard microinjection of DNA and RNA molecules into embryos. The DNA or RNA molecules encode specific zinc finger nucleases and the mutations are faithfully and efficiently transmitted through the germline.

The invention also provides a clone produced from a transgenic animal of the invention, or from any animal carrying a mutation of the invention (whether transgenic or not). These cloned animals may be produced by methods such as somatic cell nuclear transfer. This technique enables the creation of a new animal (clone) from a single somatic cell without the requirement to perform processes which occur after the fertilization of an oocyte by a sperm in a generative process. A cloned embryo produced using the technique is transferred into an estrus-synchronized surrogate mother to create a new animal. Briefly, in the somatic cell nuclear transfer process, when an immature oocyte is cultured and grown in a medium supplemented with various hormones and growth factors for 24-72 hours, it is matured to metaphase II of meiosis and is referred to as an in vitro-matured oocyte. An oocyte collected by superovulation with hormones is referred to as an in vivo-matured oocyte. The haploid of the mature oocyte produced using this method is removed by a micromanipulator, and the somatic cell of an animal to be cloned is injected into the perivitelline space or cytoplasm of the enucleated oocyte. Following this, the somatic cell injected into the perivitelline space or cytoplasm is physically fused with the enucleated oocyte by electrical stimulation. The fused oocyte is activated either by electrical stimulation or a chemical substance. The cloned embryo thus produced is transferred into the oviduct or uterus of a surrogate mother by a surgical or non-surgical procedure to allow living offspring to be born.

It will be appreciated that it is not intended to limit the invention to the above example only. Many variations, which may readily occur to a person skilled in the art, may be possible without departing from the scope thereof as defined in the accompanying claims.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention is further described in detail by reference to the following experimental example. The example is provided for illustration purposes only, and is not intended to be limiting unless otherwise specified. Thus, the invention encompasses any and all variations which become evident as a result of the teaching provided herein.

### BRIEF DESCRIPTION OF THE FIGURES

FIG. 1 shows the pedigree of Cow 363. In the pedigree drawing, all animals producing milk with an advantageous milk profile, or siring cows producing milk with an advantageous milk profile, are shaded. Males are represented as squares, females are represented as circles.

FIG. 2 shows a graph depicting the marker association map for the low milk fat content in the mapping pedigree of FIG. 1. The F-value for the association of each marker with milk fat percentage in the mapping pedigree of Cow 363 is displayed as a function of marker position on the relatively small segment spanning from chromosome 14pter to nucleotide position 2 million base pairs. High F-values indicate a high degree of association of the chromosomal region surrounding the marker with the location of the mutation responsible for the advantageous milk profile phenotype. The position of the DGAT1 gene from nucleotide positions 444 kb-446 kb is indicated by a rectangle. Only markers with all 3 genotype classes and with a count of 5 or more are depicted. The F-value of 20.188 corresponding to the false discovery rate is indicated by a dashed line.

FIG. 3 shows a graphical representation of the mutation in Cow 363 as an adenine (A) to cytosine (C) nucleotide substitution near the 3′-end of exon 16 (position 8078 in GenBank. Accession AY065621/GI:18642597). The mutation encodes: (i) a DGAT1 protein in which the methionine residue at position 435 is replaced by leucine (M435L), and (ii) a DGAT1 protein lacking the 21 amino acids encoded by exon 16 (Δ418-438). A: graphical overview of the DGAT1 gene structure. Exons are indicated as rectangles, and introns and intergenic regions are indicated by a thick line. B: the sequence chromatogram obtained from exon 16 of Cow 363 who is heterozygous at the 8078 position. C and D: Partial nucleotide and amino acid sequences surrounding the mutation in Cow 363. C: Partial sequences of the wild-type DGAT1 gene (upper sequence) and DGAT1 protein (lower sequence). Exon sequences are indicated in uppercase letter, and partial intron sequences are in lower-case letters. The exon splicing motif (ATGATG) near the 3′-end of exon 16 is underlined. Removal of sequences corresponding to intron 15 and intron 16 during RNA processing is indicated by horizontal lines within the protein sequences. D: Partial sequence of the mutated. DGAT1 gene in Cow 363 (upper sequence), partial sequence of the mutated DGAT1 protein in which the methionine residue at position 435 is replaced by leucine (middle sequence; horizontal lines indicate removal of sequences corresponding to intron 15 and intron 16 during RNA processing), and partial sequence of the mutant DGAT1 protein lacking the 21 amino acids encoded by exon 16 (lower sequence; horizontal lines indicate removal of sequences corresponding to intron 15, exon 16, and intron 16). The mutated exon splicing motif (CTGATG) in exon 16 of the DGAT1 gene is underlined. E: Mutation of the exon splicing motif results in excision of exon 16 during mRNA processing. Reverse transcriptase PCR of liver total RNA obtained from homozygous carriers of the wild-type DGAT1 gene (cows 351 and 352) shows the expected 200 base pair product containing exon 16. The 200 base pair product is less abundant in samples from cows heterozygous for the mutation at position 8078 (cows 346 and 353). The samples from cows heterozygous for the mutation at position 8078 also generate an additional PCR product of 137 base pairs, which lacks the nucleotides corresponding to exon 16. The band migrating at approximately 240 base pairs in the 346 and 353 lanes was shown to be a heteroduplex of the 137 and 200 base pair products.

FIG. 4 shows the evolutionary conservation of DGAT1 proteins in the region corresponding to the mutation in Cow 363. Partial amino acid sequences from Bos taurus (Bta, accession NP_{—}777118.2), Bos indicus (Bin, ABR27822.1), Bubalus bubalis (Bbu, ABB53651.2), Homo sapiens (Hsa, NP_{—}036211.2), Pan troglodytes (Ptr, XP_{—}520014.2), Macaca mulatta (Mmu, XP_{—}001090134.1), Canis familiaris (Cfa, XP_{—}539214.2), Equus caballus (Eca, XP_{—}001917097.1), Capra hircus, (Chi, ABD59375.1), Ovis aries (Oar, NP_{—}001103634.1), Sus scrofa (Ssc, NP_{—}999216.1), Monodelphis domestica (Mdo, XP_{—}001371565.1), Danio rerio (Dre, NP_{—}956024.1), Mus musculus (Mus, NP 034176.1), and Rattus norvegicus (Rno, NP 445889.1) are aligned and shown in contrast to the partial sequences encoded by the mutant allele (M435L or Δ418-438) of Cow 363. Dashes indicate the 21 amino acids missing in the Δ418-438 mutant protein as a result of skipping exon 16. Asterisk characters in the bottom row indicate complete conservation in the species compared.

FIG. 5 shows the effect of the Δ8078C mutation in Cow 363 on the enzymatic activity of the DGAT1 protein. Wild-type and mutant forms of DGAT1 proteins obtained by expressing the corresponding cDNAs at comparable levels (i.e. within ±10%) in baker's yeast strain H1246, which lacks endogenous diacylglycerol transferase activity, were assayed for their ability to transfer [¹⁴C]oleoyl-CoA to diacylglyceride. DGAT1-232A-Δ418-438 (SEQ ID NO:47) is encoded by the mutant DGAT1 gene of Cow 363, i.e. contains an alanine residue in position 232 and lacks the 21 amino acids encoded by exon 16 (i.e. Δ16). Full-length, wild-type proteins DGAT1-232A (SEQ ID NO:3) and DGAT1-232K (SEQ ID NO: 45) contain alanine and lysine residues in position 232, respectively. A: Thin-layer chromatogram of products obtained from diacylglycerol transferase reactions from two independent yeast transformants of each cDNA expression plasmid, and from the same host strain transformed with the expression plasmid without insert (empty vector). Positions of [¹⁴C]oleoyl-CoA (reaction substrate) and tricylglycerol (reaction product) are indicated by arrows. B: Triacylglyceride yield synthesized by recombinant DGAT1 proteins and controls was quantified by densitometric imaging of the TLC plate shown in panel A, and is depicted as means of counts per area (+standard error) from the two independent transformants of each plasmid. In all reactions, equivalent amounts of total cell extract were used.

FIG. 6 shows the effect of the A8078C mutation on diacylglyceral transferase activity in the livers of cows heterozygous for the mutation. Microsomal protein samples prepared from liver biopsies taken from wild-type (n=11, AA) and carrier cows (n=13, CA) were assayed for their ability to transfer [¹⁴C]oleoyl-CoA to diacylglyceride by thin layer chromatogaphy and scintillation counting. The mean incorporation of [¹⁴C]oleoyl-CoA into triglyceride is depicted as counts per minute (CPM) and was significantly different between wild-type and heterozygous carrier cows (AA vs. CA, p<0.05), as indicated by the asterix.

### EXAMPLE

### Analysis of the Genetic Basis for the Advantageous Milk Profile Phenotype

This example describes the identification of a cow with increased milk volume, reduced milk fat content, decreased saturated fatty acid content, increased unsaturated and omega-3 fatty acid contents, and decreased fat hardness, and the investigation of the genetic basis for these characteristics using a program conducted for the discovery of novel mutations controlling economically important milk traits.

### Materials and Methods

1. Identification of Cows with Extreme Milk Traits

Several million animals in the New Zealand national dairy herd were screened for cows producing high volumes of milk with reduced fat percentage under standard New Zealand dairy farming practices.

### 2. Analysis of Solid Fat Content and Fatty Acid Composition

Milk samples were collected during the peak lactation phase from a.m. and p.m. milkings and combined to make a single composite sample for each animal.

The solid fat content (SFC) of the extracted milk fat was determined by pulsed nuclear magnetic resonance (NMR) using a Bruker Minispec NMS 120 NMR instrument (Bruker Analytische Messtechnik GmbH, Rheinstetten, Germany) using the procedures described by MacGibbon A K H and McLennan W D, 1987, NZ J. Dairy Science and Technology, 22:143-156. The SFC results are expressed as percentage solid fat. Milk fat samples were tempered using the sequential method described in MacGibbon A K H and McLennan W D, 1987, supra), which requires melting the fat at 60° C. for 30 min, overnight crystallisation of melted samples at 0° C., followed by determination of the SFC at 5° C. intervals (after 45 min equilibration) to 40° C. The predominant value used in the comparisons was the SFC at 10° C.

The fatty acid content of the milk fat was determined by fatty acid methyl, ester (FAME) analysis (see MacGibbon A K H, 1988, NZ J. Dairy Science and Technology, 23:399-403).

The casein and whey protein content of the milk samples was determined by HPLC and SDS-PAGE as described (Mackle T T, et al., 1999, NZ J. Dairy Sci., 82:172-80).

### 3. Analysis of Heritability of Rare Milk Characteristics

Standard in vitro fertilisation techniques and embryo implantation into surrogate dams were used to produce seven female and five male offspring from Cow 363. Calves were raised as per standard New Zealand dairy farming practices, and the heifer calves were inseminated at 15 months of age.

Inter-generational transmission of the advantageous milk profile phenotype was assessed by measurement of milk fat and protein content by Fourier transformed infrared spectrometry (http://www.foss.dk) (FTIR), solid fat content, milk fatty acid composition, and milk protein composition.

### 4. Generation of a Pedigree for Mutation Mapping, and Determination of Milk Fat Phenotypes

Semen was collected from the five sons of Cow 363, and used to inseminate unrelated Holstein-Friesian cows in a commercial milking herd. Heifer calves were raised under standard New Zealand dairy farming practices and inseminated at 15 months of age.

The milk fat percentages of 101 lactating daughters of Cow 363's five sons were determined by FTIR during the early and peak lactation periods and used as phenotypes for mapping of the mutation responsible for the advantageous milk profile phenotype.

The milk fat percentages of founder Cow 363, her seven daughters (Cows 273, 351, 346, 352, 353, 354, and 357), Cow 107 (daughter of 346), Cows 108 and 307 (daughters of Cow 354), and 50 lactating daughters of the three sons of Cow 363 transmitting the advantageous milk profile phenotype were determined by FTIR during the early and peak lactation periods, and used as phenotypes to map the genome region harbouring the mutation responsible for the advantageous milk profile phenotype.

### 5. Genotyping

Genomic DNA was isolated from whole blood from 199 animals within the Cow 363 pedigree: Cow 363, her sire and grandsires, five sons, seven daughters, and Cows 107, 108, and 307, 101 granddaughters sired by her five sons, complemented by 79 dams of the granddaughters. The samples were genotyped using the Illumina BovineSNP50 Genotyping BeadChip (Illumina Inc., San Diego, Calif., U.S.A.). A total of 45,261 informative SNP markers were used for association mapping.

Genomic DNA was isolated from whole blood or semen from 185 sires frequently used for artificial insemination in the New Zealand dairy population, and from 80 sires and 1595 cows representing the BoviQuest. Friesian-Jersey crossbreed herd (Spelman R J, et al., 2001, Proc. Assoc. Advmt. Anim. Breed. Genet. 14:393-396). The samples were genotyped for the A8078C mutation in a custom-designed iPLEX™ Gold assay (SEQUENOM, San Diego, Calif., USA) using the PCR primers given in SEQ ID NO:5 and SEQ ID NO:6, and the extension primer given in SEQ ID NO:7. DNA from eight animals from the Cow 363 pedigree heterozygous for the mutation was used as positive controls.

### 6. Association Mapping

SAS version 9.1 was used to analyse the Illumina genotypes. The data were merged by SNP name with the chromosomal location data provided by Illumina. Separate datasets were created for each chromosome. The milk fat percentage measurements were merged by Illumina sample ID into the dataset. The SNP alleles were merged to create a genotype variable. These genotypes were ordered alphabetically for analysis purposes, i.e. A, AC, C and so on. These were then turned into numeric values for use in SAS (i.e. A, AC, C equated to 0, 0.25 and 0.5, and so on). In each genotype pairing the lowest order alphabetical genotype level was used as the reference/baseline, group in the GLM modelling. The genotypes were coded according to Table 2 below.

Analysis was performed separately for each chromosome. A separate generalised linear model test was performed for each SNP, i.e. PHENOTYPE=GENOTYPE for each chromosome. The following model was used for generalised linear modelling:

y=genotype+e

(where y=milk fat content (quantitative variable), genotype=SNP marker for a particular chromosome and e=error or residual).
F-values and p-values for each SNP marker were calculated by linear regression (ANOVA modelling), Identical results were obtained by adopting a 0, 1, 2 genotype coding convention for all the SNP markers. The reference group for each marker was the lowest order homozygous group, which was denoted by a 0. The heterozygous group was represented by a 1 and the highest order homozygous group was referenced by a 2.

TABLE 2

| Genotype | Genotype coding |
|---|---|
| A, AC, C | 0, 0.25, 0.5 |
| A, AG, G | 0, 1, 2 |
| A, TA/AT, T | 0, 1, 2.5 |
| G, TG, T | 2, 2.25, 2.5 |
| C, GC/CG, G | 0.5, 1, 2 |
| C, TC, T | 0.5, 1, 2.5 |

The Benjamin and Hochberg procedure was adopted to determine the false discovery rate (FDR). Only those markers with all 3 genotype classes and with a count of 5 or more were included in the calculation. The p-values for each marker returned from the association mapping were ordered in magnitude starting with the lowest p-value (i.e., the most significant marker). The largest p-value was identified by taking the threshold, such that:

P(i)≦α/(m−(i)+1)

(where α was set at 0.05; m is the total number of markers and i is the ascending order of magnitude of the p-values from the association mapping).

A total of 10,096 markers met the criteria (markers with all 3 genotype classes and with a count of 5 or more) and were included in the calculation. The calculated threshold was 4.9549103×10⁻⁶. Markers with a P₍ⱼ₎≦4.9549103×10⁻⁶ were rejected (where j is the individual marker). 6 markers were identified as having a p-value less than the threshold. The F-value threshold/FDR was created by taking the associated F-value with the largest p-value, such that P(i)≦α/(m−(i)+1). In this case, the largest p-value returned was from ARS-BFGL-NGS-18858 with a p-value of 1.1817496×10⁻⁶; the associated F-value returned from the ANOVA modelling was 20.187953515.

### 7. Candidate Gene Sequence Analysis

DGAT1 was identified as a candidate gene for the advantageous milk profile phenotype. Intron/exon boundaries were determined by homology with the human gene sequence and from the GenBank annotation of the bovine DGAT1 gene (accession AY065621.1; GI:18642597). Exon 1 was amplified from genomic DNA using the primers presented as SEQ ID NOs:8-11, exon 2 was amplified using the primers presented as SEQ ID NO:12 and SEQ ID NO:13, exon 3 was amplified using the primers presented as SEQ ID NO:14 and SEQ ID NO:15, and the chromosome segment spanning exons 4 to 17 was amplified using the primers presented as SEQ ID NO:16 and SEQ ID NO:17. Exon and intron/exon boundary sequences were determined in both directions using the primers presented as SEQ ID NOs: 18-35.

### 8. Biopsy Sampling

Liver tissue was collected by needle biopsy at various times during peak and mid-lactation. Biopsy procedures and associated animal treatment protocols were reviewed and approved by the AgResearch Ruakura Animal Ethics Committee (Hamilton, New Zealand). Tissue samples were immediately snap-frozen in liquid nitrogen and stored at −85° C. until further processing.

9. DGAT1 cDNA Cloning and Construction of Yeast Expression Vectors

DGAT1 cDNAs were amplified from total liver RNA obtained from wild-type Cow 352 and mutant Cow 354. Primers presented as SEQ ID NOs: 36 and 37 were used for the first 10 amplification cycles; followed by 20 amplification cycles with primer presented as SEQ. ID NO:38, paired with: (i) primer presented as SEQ ID NO:39 (cDNA encoding DGAT1-232K); (ii) primer presented as SEQ ID NO:37 (cDNA encoding DGAT1-232A); or (iii) primer presented as SEQ ID NO:40 (cDNA encoding DGAT1-232A-Δ418-438). Advantage GC Genomic LA Polymerase Mix (Clontech) was used for all 30 amplification cycles.

The PCR products were cloned into pCR2.1-TOPO (Invitrogen) using the TOPO TA Cloning kit (Invitrogen), and transformed into. Escherichia coli TOP10 (Invitrogen). Plasmid DNA was prepared from recombinant colonies and the sequence of the inserts was determined using standard protocols.

DGAT1 inserts were excised from pCR2.1-TOPO vectors with HindIII/NotI (DGAT1-232K and DGAT1-232A-Δ418-438) and HindIII/EcoRI (DGAT1-232A), and cloned into the HindIII and NotI or EcoRI sites of yeast expression vector pYES2 (Invitrogen) using a Rapid DNA Ligation Kit (Roche). All DNA-modifying enzymes were obtained from Roche. The nucleotide sequences of the plasmid inserts and their adjoining regions were confirmed using standard protocols.

DGAT1 expression plasmids and non-recombinant vector pYES2 were introduced into strain H1246 (Sandager L et al., 2002, J. Biol. Chem. 277:6478-6482) by electroporation as described (Ausubel et al., 1987, supra). Transformants were selected on uracil-free minimal medium containing glucose as sole fermentable carbon source (SCD-ura) by incubation at 30° C. for three days. After two rounds of cloning by limited dilution, transformants harbouring DGAT1 expression plasmids were screened by PCR using primer pairs presented as SEQ ID NOs:37 and 38, SEQ ID NOs:38 and 39, and SEQ ID NOs: 38 and 40 for the DGAT1-232A, DGAT1-232K, and DGAT1-232A-Δ418-438 alleles, respectively. The sequence of the PCR products was determined by standard methods. Recombinant yeast strains were routinely maintained on SCD-ura plates.

### 10. Heterologous Expression of DGAT1 Proteins in Baker's Yeast

The recombinant yeast strains were grown to OD600 nm 0.4-0.6 in 100 mL SCD-ura. Cells were washed once in 100 mL sterile water, and resuspended in 100 mL uracil-free synthetic complete medium containing 2% galactose as sole carbon source and incubated for 12-16 hours at 30° C. on a rotary shaker. Twenty OD600 nm of culture was harvested into a 15 mL glass centrifuge tube, and 1 OD600 nm was retained for mRNA quantification. Cells were, sedimented at 4000 g for 5 mins, washed in 1 volume of water, and resuspended in 50 μL glass bead disruption buffer (20 mM Tris-HCl (pH 7.9), 10 mM MgCl₂, 1 mM EDTA, 5% glycerol, 1 mM DTT, 0.3M ammonium sulfate, Complete protease inhibitor cocktail (Roche), 0.8 mM Pefabloc SC PLUS (Roche). 600 mg of glass beads (diameter 450-550 μm, Sigma) was added to each tube and the cells were disrupted by vigorous vortexing at 4° C. for 5 mins. 500 μL glass bead disruption buffer was added to each tube, and the lysate recovered by pipetting. The lysate was centrifuged at 12000 g⁻¹ for 10 min, and the supernatant retained. Protein concentrations of the cleared lysates were determined using the DC protein assay (Bio-Rad). Lysates were used immediately to determine diacylglycerol transferase activity, or stored at −80° C.

For quantification of DGAT1 expression in yeast transformants, 1 OD600 nm of galactose-induced yeast culture was harvested into a 1.5 mL eppendorf tube and sedimented at 4000 g for 5 minutes. Spheroplasts were prepared using the Yeast Protein Extraction Buffer Kit (GE Healthcare). Spheroplast RNA was extracted using the RNeasy kit (Qiagen). 400 ng of total RNA was transcribed into cDNA using the Superscript III First Strand Synthesis Kit (Invitrogen). Levels of recombinant DGAT1 mRNA were determined by quantitative PCR reactions using cDNA template, 2× Probes Master Mastermix (Roche), and primer pair presented as SEQ ID NOs:49 and 50. The fluorescent probe used to detect amplification was Universal Probe Library #98 (Roche)(5′-CTGTGCCT-3′). Thermal cycling conditions were as follows: Pre-Incubation 95° C. for 5 minutes, followed by 45 cycles of 95° C. (10 seconds), 60° C. (15 seconds), and 72° C. (1 second). Thermal cycling and fluorescence detection was performed on a LightCycler 480 instrument (Roche). A standard curve was derived using a dilution series of pooled cDNA samples to assess amplification efficiency. All samples and standards were measured in triplicate. Identical assays were performed to determine mRNA levels of yeast GAPDH using primers presented as SEQ ID NOs:51 and 52, and fluorescent detection probe Universal Probe Library #82 (Roche)(5′-CTCCTCTG-3′). Cycling conditions were identical to the DGAT1 assay. DGAT1 expression levels were determined by computing the ratio of mean crossing point value (Cp) for bovine DGAT1 mRNA to the mean Cp for the yeast GAPDH mRNA.

### 11. Diacylglycerol Transferase Assay of Recombinant DGAT1 Proteins

Transfer of oleoyl-CoA to diacylglycerol was quantified in 200 μL of a solution consisting of 250 mM sucrose, 1 mM EDTA, 20 mM MgCl₂, 100 mM Tris-HCl (pH 7.5), 25 μg fatty acid-free serum albumin (Sigma), 40 nmol 1,2-dioleoyl-sn-glycerol (Sigma), and 5 nmol [1-¹⁴C]oleoyl-CoA (specific activity 50-62 mCi/mmol; GE Healthcare). Clarified yeast cell lysates containing 50 μg protein were used in each reaction. The reactions were preincubated at 37° C. for two minutes. Diacylglycerol and oleoyl CoA were added and the reaction mixtures were incubated at 37° C. for a further 8 min. The reactions were stopped by adding 800 μL of chloroform:methanol (1:1) containing 15 μg/ml triolein, and mixing. 600 μL of chloroform was added to each reaction, mixed, and incubated overnight at −20° C. After adding 300 μL acidified H₂O (17 mM NaCl; 1 mM H₂SO₄), the organic phase was recovered and dried under a stream of nitrogen. The recovered material was dissolved in 20 μL acetone and separated on silica-gel 60 thin layer chromatography plates (Merck) using hexane/ethyl acetate (9:1 vol/vol). TLC plates were dried and exposed for 48 hours to a PhosphorImager screen (Kodak). TLC images were obtained by scanning with a Pharos FX plus scanner (Bio-Rad). Triacylglyceride (TAG) was identified by retention factor (Rf; migration distance of solvent divided by migration distance of TAG), previously determined with TAG standard (Sigma).

12. Preparation of Microsomes from Tissue Biopsies

300 mg of liver were homogenized in 4 ml of ice cold homogenization medium (0.25 M sucrose, 1 mM EDTA, buffered at pH 7.4 with 5 mM Tris). Homogenization was carried out on ice with a Polytron homogenizer PT1200 at a speed setting of 5, three times for approximately 10 seconds. The homogenates were centrifuged at 4° C. for 30 minutes at 15,000 g. The supernatants were recovered and centrifuged at 4° C. for 1 hour at 100,000 g. The supernatants were discarded and the pellets (microsomal fraction) resuspended in 150 μL of homogenization medium and stored at −80° C. The protein concentration was determined using a Bio-Rad protein assay.

### 13. Diacylglycerol Transferase Assay of Tissue Microsomes

Transfer of oleoyl-CoA to diacylglycerol was quantified in 100 μL of a solution consisting of 0.1 M K-phosphate buffer (pH7.4), 10 mM MgCl₂, 1 mM 1,2-dioleoyl-sn-glycerol (Sigma), and 0.2 μCi [1-¹⁴C]oleoyl-CoA (American Radiolabeled Chemicals, Inc.). Microsomal samples containing 40 μg proteins were used in each reaction. The reaction mixtures were incubated at 37° C. for 30 min. The reactions were stopped by adding 750 μL of chloroform:methanol (1:1) and mixing. After adding 375 μL acidified H₂O (17 mM NaCl, 1 mM H₂SO₄), the organic phase was recovered and dried under a stream of nitrogen. The recovered material was dissolved in 20 μL hexane and separated by on silica-gel 60 thin layer chromatography plates (Merck) using hexane:ethyl ether:acetic acid (80:20:1 vol:vol:vol). The regions corresponding to the TAG area were identified by comparison to TAG standard (Sigma), scraped off the TLC plates and transferred to 1.5 ml centrifuge tubes. 500 μL of Optifase Hisafe 3 (Perkin Elmer) cocktail were added to the tubes and the [¹⁴C] beta emissions were measured as counts per minute in a Wallac 1409 Liquid Scintillation Counter (Perkin Elmer).

### 14. Analysis of Exon 16 Splicing

To determine the presence or absence of exon 16, RNA was extracted from mammary and liver biopsies obtained from four mutant and four wild-type cows from the pedigree of Cow 363 using the Qiagen RNeasy Kit (Qiagen) according to the manufacturer's instructions. Briefly, tissue biopsies were homogenized by grinding in Qiagen buffer RLT with Fastprep Lysing matrix D columns in a Fastprep instrument (Qbiogene). RNA was eluted in RNAse-free water and quantified by absorbance at 260 nm. RNA integrity was verified by electrophoresis on a RNA 6000 nano labchip and BioAnalyzer instrument (Agilent Technologies). cDNA was prepared using an oligo dT primer and the First Strand cDNA Kit (Invitrogen) according to the manufacturer's instructions.

Primers presented as SEQ ID NOs:41 and 42 were used to amplify the region from position 1178 to position 1377 of the coding region of DGAT1 (as represented by GenBank accession NM_{—}174693.2; GI:110350684) using 1 μl tissue cDNA, Taq DNA polymerase, PCR buffer and Q solution (all from Qiagen). After 30 amplification cycles [30 sec 94° C., 30 sec 60° C., 30 sec 72° C.], PCR products were resolved in a 1.5% agarose gel according to standard protocols. Size of PCR products was determined by comparison to the 1 kb+ ladder (Invitrogen). Expected amplicon sizes were 200 and 137 basepairs for mRNAs containing or lacking exon 16, respectively.

### 15. Identification of Splicing Regulatory Motifs

To identify sequences with splicing regulatory activity, bovine DGAT1 gene sequences were submitted to the RESCUE-ESE web server (http://genes.mitedu/burgelab/rescue-ese/). RESCUE-ESE identifies splicing enhancer motifs by comparing the query sequence to hexamer sequences with experimentally validated exon regulatory activities (Fairbrother W G, et al., 2002, Science 297:1007-13). The bovine DGAT1 sequence was compared to human, mouse, and zebrafish (Danio rerio) motifs (Yeo G, et al., 2004, Proc. Natl. Acad. Sci. USA 101:15700-5).

### 16. Feed Intake Measurements

15 cows heterozygous for the A8078C mutation and 15 non-mutant cows (homozygous AA) in their second lactation Were housed indoors for 14 days during the mid-lactation phase (November) and fed fresh pasture through the use of calan gates (use of calan gates in feed intake trials with cattle has been described by Ferris et al., 2006, Irish. Journal of Agricultural Research 45: 149-156). Each cow received a freshly cut pasture diet twice daily following milkings (approx. 0900 and 1600 hours each day). Dry matter content of fresh feed was determined twice daily (a.m. and p.m.) in triplicate by drying three 150 g samples at 95° C. for 48 hours. The estimated feed allowance was 25 kg DM/cow/day of fresh pasture.

The wet weight of residuals of a.m. and p.m. rations was recorded daily for each cow, and dry matter content was determined in triplicate for each cow and ration as described above.

### Results

1. Cow 363 Produces High Volumes of Milk with Rare Fat Composition

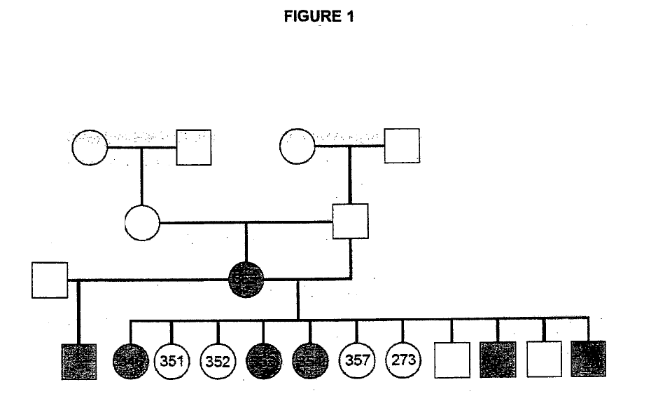

Cow 363 was identified as producing high volumes of milk with reduced fat percentage under standard New Zealand dairy farming practices. The pedigree of Cow 363 is shown in FIG. 1. All animals producing milk with an advantageous milk profile, or siring cows producing milk with an advantageous milk profile, are shaded. Males are represented as squares, females are represented as circles. As can be seen from FIG. 1, three of Cow 363's daughters (346, 353 and 354) produce milk with an advantageous milk profile, while three of her five sons have sired cows that produce milk with an advantageous milk profile.

The average fat content of milk from Cow 363 was 2.81% (standard deviation 0.1%), which is considerably lower than the national average for Holstein-Friesian cows of 4.34%.

The average fat production of Cow 363 was 175 kg per season (standard deviation 26 kg; average length of season 253 days), compared to the New Zealand Holstein-Friesian average of 162 kg (length of season 270 days).

The average milk production of Cow 363 was 6224 litres per season (standard deviation 924 litres; average length of season 253 days), which is considerably higher than the New Zealand average of Holstein-Friesian cows (4604 litres; length of season 270 days).

### 2. The Milk Characteristics of Cow 363 are Heritable

Further matings were performed to extend the pedigree shown in FIG. 1. Semen from the five sons of Cow 363 was used to produce 101 lactating female offspring. Analysis of their milks demonstrated that three of the five sons of Cow 363 have sired cows that produce low-fat milks, indicating that these bulls have inherited the genetic locus responsible for the advantageous milk profile phenotype. Additional female offspring were obtained from, the daughters having an advantageous milk profile of Cow 363. The milk fat percentages of these cows are summarized in Table 3 below.

TABLE 3
Milk fat percentages of animals in the mapping population

| Cow No | Milk fat % |
|---|---|
| 1 | 4.78 |
| 2 | 3.26 |
| 3 | 4.62 |
| 4 | 4.33 |
| 5 | 2.81 |
| 6 | 3.93 |
| 7 | 3.18 |
| 8 | 4.87 |
| 9 | 3.95 |
| 10 | 2.38 |
| 12 | 4.04 |
| 13 | 2.83 |
| 14 | 3.96 |
| 16 | 4.11 |
| 17 | 4.77 |
| 18 | 4.63 |
| 19 | 3.98 |
| 20 | 4.39 |
| 21 | 4.35 |
| 23 | 4.09 |
| 24 | 4.25 |
| 25 | 3.25 |
| 26 | 3.41 |
| 27 | 3.99 |
| 28 | 3.85 |
| 29 | 2.58 |
| 31 | 2.52 |
| 32 | 4.41 |
| 33 | 3.37 |
| 34 | 3.93 |
| 35 | 3.72 |
| 36 | 2.7 |
| 37 | 3.41 |
| 89 | 2.76 |
| 90 | 3.83 |
| 91 | 4.31 |
| 92 | 2.53 |
| 93 | 4.33 |
| 94 | 2.54 |
| 95 | 4.45 |
| 96 | 3.58 |
| 97 | 3.88 |
| 98 | 4.38 |
| 99 | 4.52 |
| 100 | 2.8 |
| 101 | 4.27 |
| 103 | 3.89 |
| 104 | 3.96 |
| 105 | 2.96 |
| 106 | 4.22 |
| 363 | 2.87 |
| 346 | 2.64 |
| 351 | 3.78 |
| 352 | 4.64 |
| 353 | 2.69 |
| 354 | 2.52 |
| 357 | 4.17 |
| 273 | 3.95 |
| 107 | 3.52 |
| 108 | 2.64 |
| 307 | 4.16 |

As seen in Table 3, average milk fat percentage determined by Fourier transformed infra red spectrometry (http://www.foss.dk) (FUR) during the early and peak lactation season is shown for founder Cow 363, her seven daughters (Cows 273, 351, 352, 353, 354, 346, and 357), Cow 107 (daughter of 346), Cows 108 and 307 (daughters of 354), and 50 lactating daughters of the three sons of 363 transmitting the advantageous milk profile phenotype. It should be noted that milk fat percentage decreases in some animals particularly early in lactation. The data presented in the table above are the averages of individual animals obtained from multiple test dates during their early lactation. Therefore, milk fat percentage in some animals is expected to decrease further as lactation progresses. This was the data used for the genetic mapping of the trait loci.

As seen in Table 3, daughters 346, 353, and 354 of Cow 363 produced milks with similarly extreme characteristics as their darn, while daughters 351, 352, and 357 produced milks similar to unrelated control cows in the same herd, and the New Zealand average of Holstein-Friesian cows. The average fat percentage of the milks from daughters 346, 353, and 354 was 2.62% (standard deviation 0.09%), while the milk fat average of daughters 351, 352, and 357 was 4.20% (standard deviation 0.43%).

The fatty acid composition of milk obtained from Cow 363 (expressed as weight percent of total fatty acids) is shown in Table 4 below, and was highly unusual.

TABLE 4
Fatty acid content of milk fat from Cow 363

| Fatty Acid | % |
|---|---|
| C4:0 | 3.70 |
| C6:0 | 2.00 |
| C8:0 | 1.31 |
| C10:0 | 3.15 |
| C10:1 | 0.31 |
| C12:0 | 3.75 |
| C13:0 branched | 0.16 |
| C12:1 | 0.08 |
| C13:0 | 0.08 |
| C14:0 branched | 0.13 |
| C14:0 | 11.67 |
| C14:1 | 0.90 |
| C15:0 iso branched | 0.42 |
| C15:0 ante-iso branched | 0.71 |
| C15:0 | 1.24 |
| C16:0 branched | 0.29 |
| C16:0 | 21.59 |
| C16:1 | 1.42 |
| C17:0 iso branched | 0.70 |
| C17:0 ante-iso branched | 0.58 |
| C17:0 | 0.68 |
| C17:1 | 0.28 |
| C18:0 | 11.33 |
| C18:1n-9 | 21.74 |
| C18:1n-7 | 5.90 |
| C18:2n-6 | 1.57 |
| C18:3n-3 | 0.95 |
| C18:2 conj.(c9, t11) | 1.48 |
| C20:0 | 0.11 |
| C20:1n-11 | 0.08 |
| C20:1n-9 | 0.00 |
| C20:2n-6 | 0.00 |
| C20:3n-6 | 0.00 |
| C20:4n-6 | 0.00 |
| C20:3n-3 | 0.00 |
| C20:4n-3 | 0.05 |
| C20:5n-3 | 0.08 |
| C22:0 | 0.00 |
| C22:1n-13, n-11 | 0.00 |
| C22:1n-9 | 0.00 |
| C22:4n-6 | 0.00 |
| C22:5n-6 | 0.00 |
| C22:5n-3 | 0.00 |
| C24:0 | 0.00 |
| C22:6n-3 | 0.00 |
| C24:1 | 0.00 |
| Unidentified fatty acids | 1.58 |

As seen in Table 4, the percentage of saturated fatty acids in milk obtained from Cow 363 is reduced significantly to 55-60% of the total fatty acid content, while the percentage of monounsaturated and polyunsaturated fatty acids is significantly increased by 13-33% compared to Holstein-Friesian cows under grass-fed management systems in New Zealand (MacGibbon A K H and Taylor M W, 2006, Composition and Structure of Milk Lipids. Advanced Dairy Chemistry; Vol. 2. Lipids, 3^{rd} ed., 1-42. Fox, P. F., and McSweeney, P. L. H., eds. Springer, N.Y.). In addition, omega-3 fatty acids, characterised by a carbon-carbon double bond in the n-3 position, were also significantly higher in milk obtained from Cow 363.

Table 5 below shows the solid fat content (SFC) of milk obtained from cows in the pedigree of Cow 363. The SFC at 10° C. of the extracted milk fats expressed as a percentage of total solid fat is shown for the founder of the pedigree (Cow 363), the average and standard deviation of her daughters 346, 353, and 354 (daughters which produce milk with an advantageous milk profile), and three unrelated control cows in the same herd (control cows) which do not produce milk with an advantageous milk profile.

TABLE 5
Solid fat content (SFC) at 10° C. of
milks obtained from cows of the Cow 363 pedigree

| Cow(s) | % SFC (10° C.) |
|---|---|
| 363 | 43.9 |
| 346, 353, 354 | |
| SFC average | 42.2 |
| Standard deviation | 2.8 |
| Control cows | |
| SFC average | 56.9 |
| Standard deviation | 4.2 |

As shown in Table 5, the milk from Cow 363 contained 43.9% solid fat at 10° C., significantly less than average cows on forage-based diets (57.7%, standard deviation 3.3%). Furthermore, the average solid fat content at 10° C. of milks from daughters 346, 353, and 354 was 42.2% (standard deviation 2.8%), while the average of unrelated control cows in the same herd was 56.9% (standard deviation 4.2%).

Table 6 below shows the fatty acid composition of milks obtained from cows of the Cow 363 pedigree. Individual fatty acids, as determined by fatty acid methyl ester analysis, are grouped and expressed as weight percent of total fatty acids. Results are shown for the founder of the pedigree (Cow 363), the average and standard deviation of her daughters 346, 353, and 354, daughters 351, 352, and 357, and three unrelated control cows in the same herd (control cows). Fatty acid groups are comprised of: Saturated fatty acids: C4:0, C6:0, C8:0, C10:0, C12:0, C13:0, C14:0, C15:0, C16:0, C17:0, C18:0, C20:0, C22:0, and C24:0; Monounsaturated fatty acids (MUFA): C10:1, C12:1, C14:1, C16:1, C17:1, C18:1n-9, C20:1n-11, C20:1n-9, C22:1n-9, and C24:1; Polyunsaturated fatty acids (PUFA): C18:2n-6, C18:3n-6, C20:2n-6, C20:3n-6, C20:4n-6, C20:3n-3, C20:4n-3, C20:5n-3, C22:4n-6, C22:5n-6, C22:5n-3, and C22:6n-3; Unsaturated fatty acids: MUFA+PUFA; Omega-3 fatty acids: C18:3n-3, C20:5n-3, and C22:6n-3.

TABLE 6
Fatty acid composition of milks obtained
from cows of the Cow 363 pedigree

| Cow(s) | Saturated | MUFA | PUFA | Unsaturated | Omega-3 |
|---|---|---|---|---|---|
| 363 | 60.61 | 24.80 | 2.65 | 27.45 | 1.03 |
| 346, 353, 354 | | | | | |
| Average | 54.25 | 30.09 | 3.22 | 33.31 | 1.33 |
| Standard | 2.08 | 1.43 | 0.14 | 1.33 | 0.11 |
| deviation | | | | | |
| 351, 352, 357 | | | | | |
| Average | 64.47 | 21.84 | 2.31 | 24.16 | 0.83 |
| Standard | 2.97 | 2.52 | 0.18 | 2.65 | 0.05 |
| deviation | | | | | |
| Control cows | | | | | |
| Average | 67.70 | 21.20 | 2.35 | 23.56 | 0.94 |
| Standard | 2.45 | 0.81 | 0.10 | 0.79 | 0.05 |
| deviation | | | | | |

As shown in Table 6, milks from daughters 346, 353, and 354 contained, on average, 54.25% saturated fatty acids (standard deviation 2.08%), 30.09% monounsaturated fatty acids (standard deviation 1.43%), and 3.22% polyunsaturated fatty acids (standard deviation 0.14%). Omega-3 fatty acid content was 1.33% (standard deviation 0.11%). In milks from daughters 351, 352, and 357, these fatty acid groups were found in similar percentage as in unrelated control cows in the same herd and the breed average.

The percentage and composition of casein and whey proteins of Cow 363 and her six daughters were within the normal variation of Holstein-Friesian cows under New Zealand pasture-based management systems.

Three of the five sons of Cow 363 sired daughters producing milks with similar advantageous milk profile phenotypes as Cow 363.

### 3. Identification of DGAT1 as a Candidate Gene and Detection of a Novel Mutation.

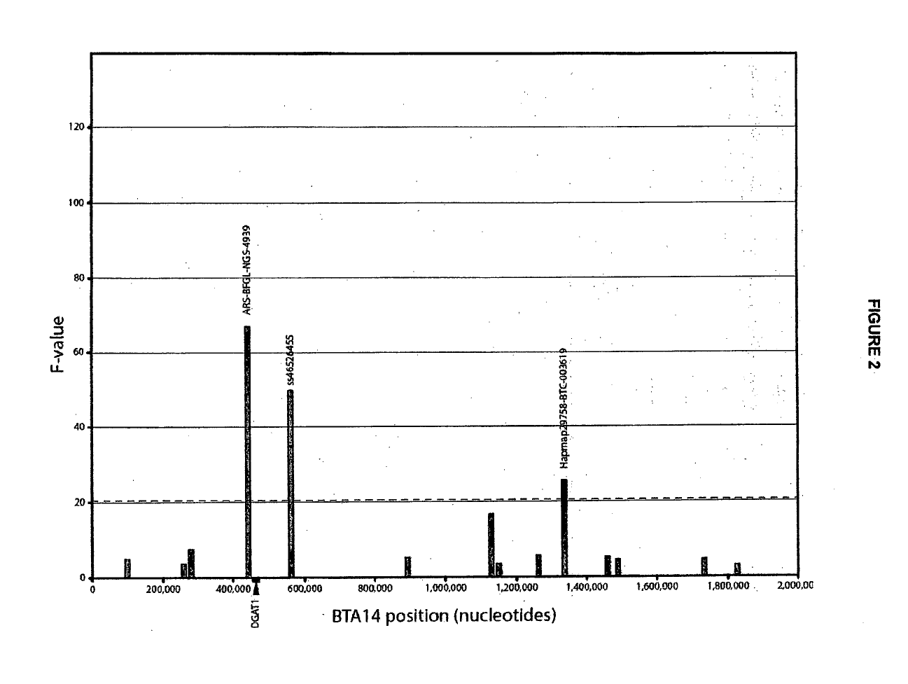

The results of the genotyping and association mapping are shown in FIG. 2. Association mapping identified SNP markers ARS-BFGL-NGS-4939, Hapmap52798-ss46526455, and Hapmap29758-BTC-003619 on chromosome 14 in the region 300-1,400 kilobases (kb) in strong association with the advantageous milk profile phenotype. Additional markers with strong association identified by association mapping are BFGL-NGS-18858, Hapmap24717-BTC-002824, and Hapmap24718-BTC-002945. These markers are mapped to a contig (Chr.Un.004.115) that was not assigned to a chromosome in the bovine genome assembly (ftp://ftp.hgsc.bcm.tmc.edu/pub/data/Btaurus/fasta/Btau20070913-freeze/).

The region spanning 1-1,400 kb on bovine chromosome 14 was identified as homologous to the region spanning nucleotide positions 142,200-146,200 kb on human chromosome 8 by BLASTN analysis (Altschul S F et al., 1990, J. Mol. Biol. 215:403-10). However, the bovine chromosome 14 sequence lacked sequences similar to human nucleotides 143,960-144,144 kb. When tested by BLASTN comparison, this gap in the inter-species alignment was closed by the sequence in bovine contigs Chr.Un.004.209 and Chr.Un.004.115. Thus these contigs, and consequently markers BFGL-NGS-18858, Hapmap24717-BTC-002824, and Hapmap24718-BTC-002945, map to the candidate region for the advantageous milk profile phenotype.

Analysis of the genes present in the candidate region identified the bovine DGAT1 gene spanning the region 444-447 kb on chromosome 14. DGAT1 encodes diacylglycerol O-acyltransferase 1 (EC 2.3.1.20) which catalyzes the terminal step in triglyceride synthesis (Cases S et al., 1998, PNAS 95:13018-23), namely the attachment of fatty acids to the glycerol backbone.

To determine whether the DGAT1 gene contained novel mutations explaining the observed advantageous milk profile phenotype, the sequence of the coding and the intron/exon boundary regions were determined in Cows 363 and 346 (advantageous milk profile including a low fat percentage), and compared to the DGAT1 sequence deposited in GenBank (accession AY065621.1; GI:18642597), and to sequences obtained from Cows 351 and 357 (normal fat percentage).

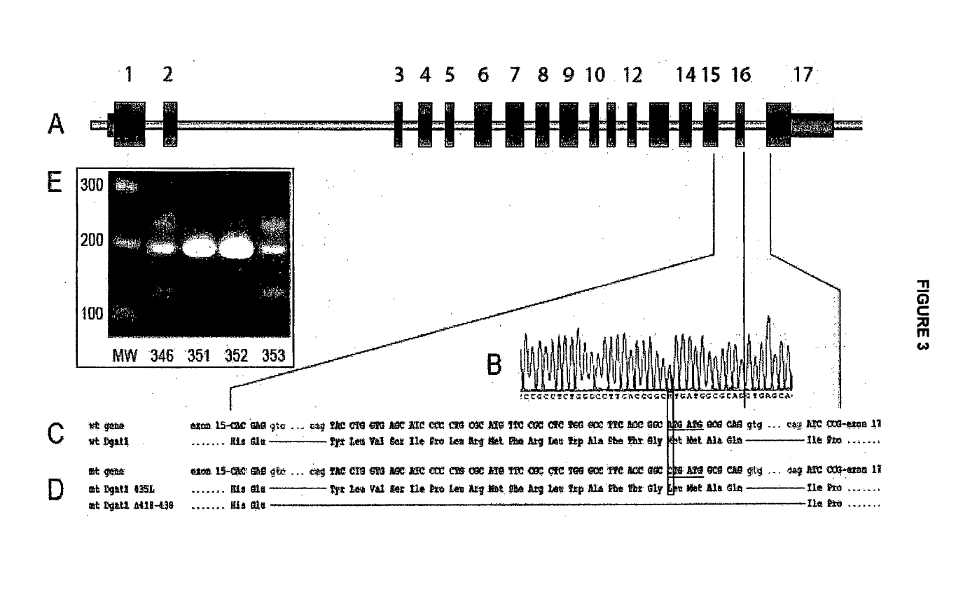

An adenine (A) to cytosine (C) nucleotide substitution in exon 16 of the DGAT1 gene (position 8078 of GenBank accession AY065621.1; GI:18642597) was heterozygous (AC) in Cows 363 and 346, and homozygous AA in cows 351 and 357 (see SEQ ID NOs:1 and 43, and SEQ ID NOs:2 and 44 for the wild-type and mutant coding regions, respectively). FIG. 3A shows the intron/exon structure of the bovine DGAT1 gene, while FIGS. 3B, 3C and 3D show the sequence surrounding the A to C nucleotide substitution.

To determine whether the mutation segregates with the advantageous milk profile phenotype in the Cow 363 pedigree; the sequence of exon 16 was determined in the sire and grandsires of Cow 363, her five sons, four remaining daughters, Cows 107, 108, and 307, and all granddaughters sired by the sons transmitting the phenotype. The A8078C mutation was found in all cows displaying the advantageous milk profile phenotype, and in the three sons of Cow 363 that sired daughters producing milks similar to that of Cow 363. The A8078C mutation was absent in the sire and grandsires of Cow 363.

The A8078C mutation was absent in 185 sires frequently used for artificial insemination in the New Zealand dairy population, and from 80 sire and 1595 cows representing the BoviQuest Friesian-Jersey crossbreed herd (Spelman et al., 2001, supra)

No mammalian DGAT1 nucleotide sequence having the A8078C mutation has been deposited in GenBank, demonstrating the novelty of the sequence from Cow 363.

The presence of the A8078C mutation restricted to the pedigree of Cow 363, its absence in her sire and maternal grandsire, and the absence of the advantageous milk profile phenotype in the darn of Cow 363 indicates that the A8078C substitution is a de novo mutation in Cow 363.

Analysis of mRNA from mammary gland and liver of cows heterozygous for the A8078C mutation revealed the additional presence of a novel, shorter DGAT1 transcript, lacking the 63 nucleotides encoded by exon 16 (FIG. 3E). In heterozygous cows, approximately half of the mammary and hepatic DGAT1 transcripts lack exon 16, indicating that the A8078C mutation efficiently disrupts splicing of exon 16 (FIG. 3D). No compensatory increase of transcription from the wild-type DGAT1 allele was detected in heterozygous cows (FIG. 3E). Transcripts lacking exon 16 were only observed in animals carrying the A8078C mutation, and could not be found in other cows.

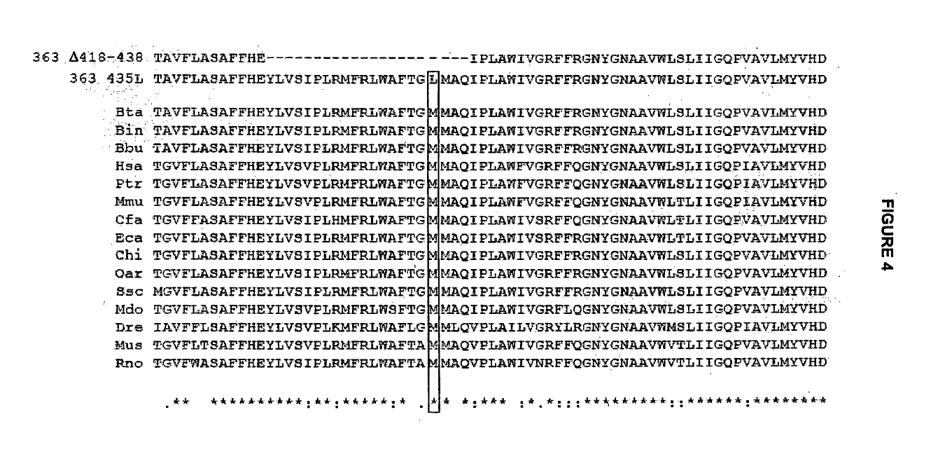

It is possible that exon 16 is correctly spliced in a small subset of mutant pre-mRNA molecules. The resulting full-length, mature mRNAs encode a mutant DGAT1 protein in which the highly conserved methionine in position 435 is replaced with a leucine residue (FIGS. 3 and 4). This non-synonymous mutation is likely to affect the catalytic properties of the enzyme.

No vertebrate DGAT1 cDNA, EST, or protein sequences lacking the region homologous to bovine exon 16 or lacking the highly conserved 21 amino acids encoded by exon 16, have been deposited in GenBank (see also FIG. 4).

Analysis of the wild-type bovine DGAT1 gene identified a putative exonic splicing enhancer motif (ESE) near the 3′-end of exon 16. The putative ESE is conserved in higher vertebrates (8078-ATGATG-8083) (see FIG. 3 and FIG. 4).

ESE motifs are short, functional cis-regulatory sequence elements adjoining intron-exon boundaries. Sequence-specific recruitment of splicing regulatory factors to ESE's assists in the identification of exons by the spliceosome during intron removal from the pre-mRNA transcript (Cartegni L et al., 2002, Nat. Rev. Genet. 3:285-298; Black D L, 2003, Annu. Rev. Biochem. 72:291-336). Mutations disrupting ESE motifs can modulate exon identification and reduce splicing efficiency, resulting in the exclusion of the entire exon from the mature mRNA transcript (Pfarr N et al., 2005, J. Immunol. 174:4172-4177; Steiner B et al., 2004, Hum. Mutat. 24:120-129).

The putative ESE identified near the 3′-end of the bovine DGAT1 gene is disrupted by the A8078C mutation in Cow 363 (8078-CTGATG-8083) (FIG. 3).

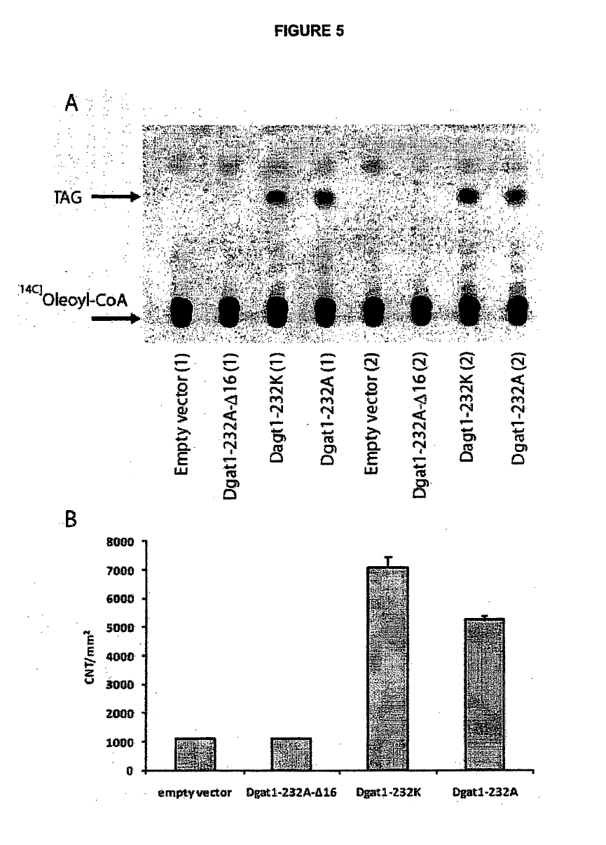

In recombinant yeast strains expressing equivalent levels of DGAT1 mRNAs, no diacylglycerol transferase activity was detectable in the recombinant mutant bovine DGAT1 protein lacking the 21 amino acids encoded by exon 16. In contrast, diacylglycerol transferase activity was readily detectable when the full-length wild-type proteins were expressed under identical conditions (FIG. 5).

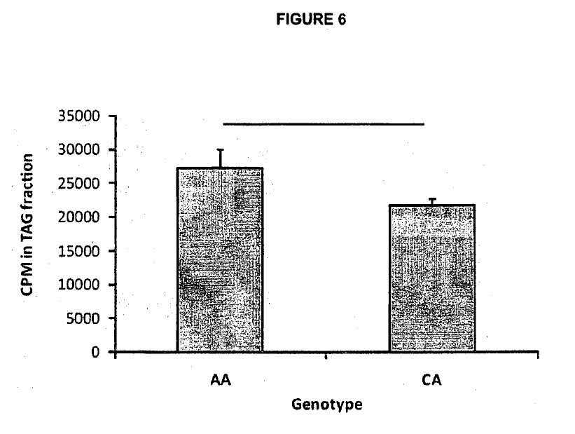

Microsomal protein preparations from liver biopsies from heterozygote mutant cows (n=13) showed a substantial (20%±4.5% mean±SEM) and statistically significant (p<0.05) reduction in [¹⁴C]oleoyl-CoA incorporation into triacylglyceride compared to equivalent microsome preparations from homozygous wild-type cows (n=11) (FIG. 6). This demonstrates that the A8078C mutation reduces diacylglycerol transferase activity in the livers of mutant cows.

Inhibition of milk fat synthesis in cows has been shown to result in increases to milk volume and milk protein yield. For example, milk fat depression induced by supplementation of pasture diets with trans-10, cis-12 conjugated linoleic acid is accompanied by increases in milk volume and milk protein yield (Griinari J M and Bauman D E, 2003: Update on theories of diet-induced milk fat depression and potential applications. Pages 115-156 in Recent Advances in Animal Nutrition. P. C. Garnsworthy and J. Wiseman, ed. Nottingham University Press, Nottingham, UK; Back P J and Lopez-Villalobos N, 2004, Proc. NZ Society of Animal Production 64:150-153). The effect of CLA on milk fat synthesis is thought to be through an effect on DGAT1 activity as CLA treatment of bovine mammary cells did not change DGAT1 expression in vitro (Sorensen et al., 2008, Lipids 43:903-912), although DGAT activity and synthesis was inhibited. Similarly, the decreased fat percentage of milk from carriers of the DGAT1 232A polymorphism is paralleled by an increase in milk volume and milk protein yield (Grisart B et al., 2004, PNAS 101:2398-2403).

These observations demonstrate that the discovery of the mutation in Cow 363 was unexpected and surprising. The mutation results in the erroneous exclusion of exon 16 from the majority of mature DGAT1 mRNA transcript molecules. The DGAT1 protein encoded by the mutant mRNA lacks 21 amino acids highly conserved through vertebrate evolution, and has no detectable fatty acyl-CoA:diacylglycerol transferase activity. The resulting reduction of trigyceride synthesis readily explains the milk fat and protein phenotypes observed in cows carrying this mutation.

### 4. Feed Requirements of Mutant Cows are Similar to Wild-Type Cows

Voluntary dry matter intake of wild-type cows (homozygous AA at position 8078) over a 14-day period was 16.3±0.3 kg of dry matter per day (mean±SEM). For the same period, cows heterozygous for the A8078C mutation consumed 16.1±0.3 kg DM/day (mean±SEM).

### Discussion

The present invention recognises that mutations in the DGAT1 gene, as described above, alone or together with polymorphisms in linkage, or in linkage disequilibrium, with it, are useful as a selection tool for animals with an advantageous milk profile, an advantageous tissue profile, and/or an increased growth rate, or animals which are capable of producing offspring with an advantageous milk profile, an advantageous tissue profile, and/or an increased growth rate. Such a strategy will enable the production of superior tissue products, particularly meat, and superior dairy products from optimised milk compositions.

1-167. (canceled)

168. An isolated nucleic acid molecule, the nucleic acid molecule comprising a DGAT1 nucleotide sequence encoding a DGAT1 protein, or a portion thereof, wherein the nucleic acid molecule has a mutation in a region of the DGAT1 nucleotide sequence equivalent to exon 16 of a bovine DGAT1 gene.

169. The isolated nucleic acid molecule according to claim 168, wherein the mutation disrupts the function of the DGAT1 protein, disrupts the expression of a full-length DGAT1 protein, disrupts the activity of the DGAT1 protein, and/or disrupts an exon splicing motif in the DGAT1 nucleotide sequence.

170. The isolated nucleic acid molecule according to claim 168, wherein the DGAT1 nucleotide sequence encodes a bovine DGAT1 protein.

171. The isolated nucleic acid molecule according to claim 170, wherein the bovine DGAT1 protein is missing one or more amino acids which are encoded by exon 16 of the bovine DGAT1 gene, or has an amino acid substitution in one or more amino acids which are encoded by exon 16 of the bovine DGAT1 gene.

172. The isolated nucleic acid molecule according to claim 168, wherein the mutation is a nucleotide substitution at position 8078 of the bovine DGAT1 gene as represented by GenBank Accession AY065621/GI:18642597.

173. The isolated nucleic acid molecule according to claim 172, wherein the mutation is an A to C nucleotide substitution at position 8078 of the bovine DGAT1 gene as represented by GenBank Accession AY065621/GI:18642597.

174. The isolated nucleic acid molecule according to claim 173, comprising the nucleotide sequence set forth in SEQ ID NOs: 2 or 44.

175. The isolated polypeptide, the polypeptide comprising a DGAT1 amino acid sequence, wherein the polypeptide has a mutation in a region of the DGAT1 amino acid sequence equivalent to the amino acids encoded by exon 16 of a bovine DGAT1 gene.

176. The isolated polypeptide according to claim 175, wherein the mutation disrupts the function of the polypeptide, disrupts the activity of the polypeptide, and/or disrupts the expression of a full-length DGAT1 polypeptide.

177. The isolated polypeptide according to claim 175, wherein the polypeptide is a bovine DGAT1 protein.

178. The isolated polypeptide according to claim 177, wherein the bovine DGAT1 protein is missing one or more amino acids which are encoded by exon 16 of the bovine DGAT1 gene, or has an amino acid substitution in one or more amino acids which are encoded by exon 16 of the bovine DGAT1 gene.

179. The isolated polypeptide according to claim 178, comprising the amino acid sequence set forth in SEQ ID NOs: 4, 46, 47 or 48.

180. A method of assessing the genetic merit of an animal with respect to an advantageous milk profile, an advantageous tissue profile, an advantageous colostrum profile, and/or an increased growth rate, the method including:
(a) determining if the animal comprises a nucleic acid molecule according to claim 168;
(b) determining if the animal comprises a polypeptide according to claim 175; and/or
(c) determining a DGAT1 exon 16 allelic profile of said animal.

181. The method according to claim 180, wherein the advantageous milk profile is selected from one or more of a reduction in total milk fat as a percentage of whole milk, an increase in the percentage of unsaturated fatty acids in the total milk fatty acid content, a decrease in the percentage of saturated fatty acids in the total milk fatty acid content, an increase in protein yield, an increase in the percentage of omega-3 fatty acids in the total milk fatty acid content, a decreased fat hardness as indicated by a reduced solid fat content of extracted milk fat at 10° C., a decrease in the ratio of milk fat:protein, an increase in volume of milk produced, and an increase in lactose yield, when compared to an animal of the same breed not carrying the mutation.

182. The method according to claim 180, wherein determining the DGAT1 exon 16 allelic profile of said animal includes:
(a) determining a presence or absence of a nucleic acid molecule according to claim 168; and/or
(b) determining a presence or absence of a DGAT1 polypeptide according to claim 175.

183. The method according to claim 182, wherein the DGAT1 exon 16 allelic profile is determined using a polymorphism in linkage or in linkage disequilibrium with a DGAT1 exon 16 allele.

184. The method according to claim 183, wherein the polymorphism in linkage or in linkage disequilibrium with the DGAT1 exon 16 allele are on bovine chromosome 14 and are selected from the group consisting of ARS-BFGL-NGS-4939, Hapmap52798-ss46526455, Hapmap29758-BTC-003619, BFGL-NGS-18858, Hapmap24717-BTC-002824, and Hapmap24718-BTC-002945.

185. The method according to claim 180, further comprising selecting the animal on the basis of the determination.

186. A method for selecting an animal that produces an advantageous milk profile, an advantageous tissue profile, an advantageous colostrum profile, and/or an increased growth rate, or an animal capable of producing progeny that produce an advantageous milk profile, an advantageous tissue profile, an advantageous colostrum profile, and/or an increased growth rate, the method including:
(a) determining if the animal comprises a nucleic acid molecule according to claim 168, and/or comprises a polypeptide according to claim 175; and
(b) selecting the animal on the basis of the determination.

187. The method according to claim 186, wherein the advantageous milk profile is selected from one or more of a reduction in total milk fat as a percentage of whole milk, an increase in the percentage of unsaturated fatty acids in the total milk fatty acid content, a decrease in the percentage of saturated fatty acids in the total milk fatty acid content, an increase in protein yield, an increase in the percentage of omega-3 fatty acids in the total milk fatty acid content, a decreased fat hardness as indicated by a reduced solid fat content of extracted milk fat at 10° C., a decrease in the ratio of milk fat:protein, an increase in volume of milk produced, and an increase in lactose yield, when compared to an animal of the same breed not carrying the mutation.

188. A method for assessing the genetic merit of an animal with respect to an advantageous milk profile, an advantageous tissue profile, an advantageous colostrum profile, and/or an increased growth rate, the method including:
(a) determining if the animal comprises a nucleic acid molecule encoding:
(i) a polypeptide (A) having biological activity of wild-type DGAT1; or
(ii) a polypeptide (B) according to claim 175; or
(iii) polypeptide A and polypeptide B,
wherein absence of a nucleic acid molecule encoding polypeptide A and presence of a nucleic acid molecule encoding polypeptide B, or presence of both a nucleic acid molecule encoding polypeptide A and a nucleic acid molecule encoding polypeptide B, indicates an advantageous milk profile, an advantageous tissue profile, an advantageous colostrum profile, and/or an increased growth rate; and/or
(b) determining if the animal comprises:
(i) a polypeptide (A) having biological activity of wild-type DGAT1; or
(ii) a polypeptide (B) according to claim 175; or
(iii) polypeptide A and polypeptide B,
wherein absence of polypeptide A and presence of polypeptide B, or presence of both polypeptide A and polypeptide B, indicates an advantageous milk profile, an advantageous tissue profile, an advantageous colostrum profile, and/or an increased growth rate.

189. A method for determining a DGAT1 genotype of an animal, the method including:
(a) determining if a nucleic acid molecule obtained from the animal is:
(i) a nucleic acid molecule (A) encoding a polypeptide having biological activity of wild-type DGAT1; or
(ii) a nucleic acid molecule (B) according to claim 168; and/or
(b) determining if a polypeptide obtained from the animal is:
(i) a polypeptide (A) having biological activity of wild-type DGAT1; or
(ii) a polypeptide (B) according to claim 175,
wherein the nucleic acid molecule or polypeptide obtained from the animal is uncontaminated by heterologous nucleic acid or polypeptide.

190. A method of assessing the genetic merit of an animal with respect to an advantageous milk profile, an advantageous tissue profile, an advantageous colostrum profile, and/or an increased growth rate, the method including:
(a) determining the presence or absence of an A nucleotide at position 8078 of the bovine DGAT1 gene as represented by GenBank Accession AY065621/GI:18642597;
(b) determining the presence or absence of a C nucleotide at position 8078 of the bovine DGAT1 gene as represented by GenBank Accession AY065621/GI:18642597;
(c) determining the presence or absence of a CC genotype at position 8078 of the bovine DGAT1 gene as represented by GenBank Accession AY065621/GI:18642597;
(d) determining the presence or absence of an AC genotype at position 8078 of the bovine DGAT1 gene as represented by GenBank Accession AY065621/GI:18642597; and/or
(e) determining the presence or absence of an AA genotype at position 8078 of the bovine DGAT1 gene as represented by GenBank Accession AY065621/GI:18642597

191. A method for selecting an animal with a DGAT1 exon 16 allelic profile indicative of an advantageous milk profile, an advantageous tissue profile, an advantageous colostrum profile, and/or an increased growth rate, the method including:
(a) (i) determining the absence of an AA genotype at position 8078 of the bovine DGAT1 gene as represented by GenBank Accession AY065621/GI:18642597; and/or
(ii) determining the absence of an A nucleotide at position 8078 of the bovine DGAT1 gene as represented by GenBank Accession AY065621/GI:18642597; and/or
(iii) determining the presence of a C nucleotide at position 8078 of the bovine DGAT1 gene as represented by GenBank Accession AY065621/GI:18642597; and/or
(iv) determining the presence of a CC genotype at position 8078 of the bovine DGAT1 gene as represented by GenBank Accession AY065621/GI:18642597; and/or
(v) determining the presence of an AC genotype at position 8078 of the bovine DGAT1 gene as represented by GenBank Accession AY065621/GI:18642597; and
(b) selecting the animal on the basis of the determination.

192. A transgenic non-human animal comprising a nucleic acid molecule according to claim 168, or a portion thereof.

193. The transgenic non-human animal according to claim 192, wherein the animal produces milk, or is capable of producing progeny that produces milk, having one or more qualities selected from the group consisting of a reduction in total milk fat as a percentage of whole milk, an increase in the percentage of unsaturated fatty acids in the total milk fatty acid content, a decrease in the percentage of saturated fatty acids in the total milk fatty acid content, an increase in protein yield, an increase in the percentage of omega-3 fatty acids in the total milk fatty acid content, a decreased fat hardness as indicated by a reduced solid fat content of extracted milk fat at 10° C., a decrease in the ratio of milk fat:protein, an increase in volume of milk produced, and an increase in lactose yield, when compared to an animal or bovine of the same breed not carrying the mutation.

194. The transgenic animal according to claim 193, which produces at least 6000 litres of milk in a season, which produces milk with less than about 3% total milk fat, which produces milk with at least about 27% unsaturated fatty acids in the total milk fatty acid content, which produces milk with less than about 57% saturated fatty acids in the total milk fatty acid content, and/or which produces milk with at least about 1.2% of omega-3 fatty acids in the total milk fatty acid content.
